(19) United States
(12) Patent Application Publication
(10) Pub. No.: US 2026/0224215 A1
(43) Pub. Date: Aug. 6, 2026

(54) SUTURE DEVICE AND RELATED METHODS

(71) Applicant: NeuroFine Corp., Miramar, FL (US)

(72) Inventors: Brad D. AURILIA, Coral Springs, FL (US); Nicholas SHATTUCK, Lauderhill, FL (US); Timothy MCSWEENEY, South Miami, FL (US); Quinton WIEBE, Davie, FL (US)

(21) Appl. No.: 19/636,745

(22) Filed: Apr. 1, 2026

Related U.S. Application Data
(63) Continuation of application No. PCT/US2024/049546, filed on Oct. 2, 2024.
(60) Provisional application No. 63/541,988, filed on Oct. 2, 2023.

(51) Int. Cl. A61B 17/04; A61B 17/00

(52) U.S. Cl. CPC A61B 17/0469; A61B 2017/00367; A61B 2017/00876; A61B 2017/047; A61B 2017/0472; A61B 2017/0474; A61B 2017/0475

(57) ABSTRACT

A suture device (100) includes a tensile member (250), a suture knot (264) associated with the tensile member (250), and a needle guide (210). The needle guide (210) includes a first needle (212) coupled to the tensile member (250), a second needle (214), and a suture knot recess (254) configured to accept the suture knot (264). At least a portion of the suture device (100) is configured to be directed into an opening in a tissue. The first and second needles (212, 214) are configured to be directed through the tissue adjacent to the opening to direct the tensile member (250) through the tissue. The tensile member (250) is configured to be pulled through the tissue when at least one of the first or second needles (212, 214) is retracted. The suture knot (264) is configured to be deployed from the suture knot recess (254) and along the tensile member (250) and tightened to close and seal the opening.

### CROSS REFERENCE TO RELATED APPLICATIONS

This application is a continuation of International Application No. PCT/US2024/049546, filed on Oct. 2, 2024, which claims priority to U.S. Provisional Patent Application Ser. No. 63/541,988, filed Oct. 2, 2023, the disclosure of each of which is incorporated herein by reference in its entirety.

This application is also generally related to U.S. Provisional Application Ser. No. 63/124,400 filed on Dec. 11, 2020 (expired), U.S. Provisional Application Ser. No. 63/191,017 filed on May 20, 2021 (expired), U.S. patent application Ser. No. 17/546,947 filed on Dec. 9, 2021, (now U.S. Pat. No. 12,035,905 issued July 16, 2024), U.S. patent application Ser. No. 17/546,958 filed on Dec. 9, 2021 (now U.S. Pat. No. 12,048,429 issued Jul. 30, 2024), PCT Patent Application No. PCT/US 2021/062667 filed on Dec. 9, 2021 (expired), PCT Patent Application No. PCT/US 2021/062671 filed on Dec. 9, 2021 (expired), U.S. Provisional Application Ser. No. 63/422,345 filed on Nov. 3, 2022 (expired), U.S. Provisional Application Ser. No. 63/431,366 filed on Dec. 9, 2022 (expired), PCT Patent Application No. PCT/US2023/036651 filed on Nov. 2, 2023 (pending), U.S. patent application Ser. No. 18/669,040 filed on May 20, 2024 (pending), and U.S. patent application Ser. No. 18/751,896 filed on Jun. 24, 2024 (pending), the disclosures of which are incorporated herein by reference in their entirety.

### TECHNICAL FIELD

Embodiments relate generally to devices, systems, and related methods for closing punctures or other openings in blood vessels and other tissues.

### BACKGROUND

Vascular access for neuro-therapy is traditionally performed through the femoral artery or by using a radial access approach. For vascular access to the brain using a traditional approach, neuro-therapy devices must navigate through lengthy tortuous segments of the anatomy to gain access to the therapeutic site. The Direct Carotid Artery Puncture (DCP) method of accessing the common carotid artery (CCA) allows doctors quicker access to the brain and eliminates the need for devices that must traverse the typical femoral interventional track. The DCP method includes a percutaneous puncture in the skin and arterial vessel to access the CCA.

Currently, to close and seal a direct carotid artery puncture, doctors may apply a suture manually prior to accessing the common carotid artery or may simply apply direct pressure to the site after intervention until the vessel is sealed. Applying direct pressure to the site relies on blood coagulation at the puncture site and may be ineffective because of the lack of suitable anatomy in the vicinity of the common carotid artery against which the pressure may be applied. The time period necessary for closing the puncture with direct pressure may also be quite lengthy. Moreover, the common carotid artery carries blood at a high pressure (100 to 200 mmHg) which further complicates the ability to effectively close the carotid puncture with current methods.

Accordingly, and despite the various advances already made in this field, there is a need for further improvements related to devices, systems and methods for sealing punctures or other openings in blood vessels and other tissues.

### SUMMARY

Generally, a suture device is provided herein. The suture device includes a tensile member, a suture knot associated with the tensile member, and a needle guide. The needle guide includes a first needle coupled to the tensile member, a second needle, and a suture knot recess configured to accept the suture knot. At least a portion of the suture device is configured to be directed into an opening in a tissue. The first and second needles are configured to be directed through the tissue adjacent to the opening to direct the tensile member through the tissue. The tensile member is configured to be pulled through the tissue when at least one of the first or second needles is retracted. The suture knot is configured to be deployed from the suture knot recess and along the tensile member and tightened to close and seal the opening.

In some embodiments, the suture knot may be deployed when at least one of the first or second needles is retracted. The tensile member may be drawn through the suture knot when at least one of the first or second needles is retracted. The suture device may include a suture knot sleeve. The suture knot may be wrapped around the suture knot sleeve. The suture knot sleeve may be at least partially inside the suture knot recess. The suture knot sleeve may retain the suture knot until the suture knot is deployed. The needle guide may include a tensile member groove configured to accommodate the tensile member. The tensile member may be deployed from the tensile member groove when at least one of the first or second needles is directed through the tissue. The tensile member groove may extend from a first needle side to a second needle side of the needle guide. The suture knot may be a sliding knot. The suture knot may be a “Tennessee Slider” knot.

In alternative embodiments, the suture device may include a handle located at a proximal end portion of the suture device, an elongated shaft coupled between the handle and the needle guide, and a needle actuator located on the handle. The needle actuator may be coupled to at least one of the first or second needles, and configured to direct at least one of the first or second needles through the tissue when the needle actuator is activated. The needle actuator may include a biasing member configured to retract at least one of the first or second needles. The needle actuator biasing member may be a spring.

In alternative or additional aspects, the needle actuator may be coupled to the first and second needles. The needle actuator may be in a first needle actuator position prior to directing the first and second needles through the tissue. The needle actuator may be configured to be moved to a second needle actuator position to direct the first and second needles through the tissue. The needle actuator may be configured to be moved to a third needle actuator position to retract the first and second needles. The needle actuator may include a plunger and a biasing member. The needle actuator may move from the first needle actuator position to the second needle actuator position when the plunger is depressed. The needle actuator biasing member may move the needle actuator from the second needle actuator position to the third needle actuator position when the plunger is released. The needle actuator biasing member may be a spring. The needle actuator may include a sprag. The sprag may move from a first sprag orientation to a second sprag orientation when the plunger is depressed. The sprag may allow the needle actuator to move from the second needle actuator position to the third needle actuator position when the plunger is released and the sprag is in the second sprag orientation. The sprag may be configured to rotate from the first sprag orientation to the second sprag orientation. The sprag may engage a first sprag groove when the needle actuator is in the first needle actuator position. The sprag may engage a second sprag groove when the plunger is depressed thereby allowing the needle actuator to move from the second needle actuator position to the third needle actuator position when the plunger is released.

In some embodiments, the needle actuator may include a catch. When the plunger is depressed, the plunger may move the catch, and when moved, the catch may allow the needle actuator to move from the second needle actuator position to the third needle actuator position when the plunger is released. At least a portion of the catch may be magnetic. The catch may engage a catch groove when the needle actuator is in the first needle actuator position, and the catch may engage a catch recess when the plunger is depressed thereby allowing the needle actuator to move from the second needle actuator position to the third needle actuator position when the plunger is released. The needle actuator may include a ferromagnetic portion. At least a portion of the catch may be magnetic, and the catch may be drawn into the catch recess by the ferromagnetic portion.

In alternative or additional aspects, the suture device may include a coupling member configured to pull the tensile member through the tissue and having first and second ends, the first end capable of being coupled to the first needle and the second end capable of being coupled to the second needle. At least one of the first or second needles may include a barbed needle tip and the coupling member may include at least one tab configured to grip the barbed needle tip. The coupling member may include a plurality of notches configured to allow the coupling member to deform. The notches may be spaced along the length of the coupling member and the spacing of the notches may at least partially define a bend radius of the coupling member. The notches may have a width and the width of the notches may at least partially define a bend radius of the coupling member. The notches may have a plurality of widths and the widths of the notches may at least partially define a plurality of bend radii of the coupling member. The notches may be configured to allow the coupling member to deform in a single plane.

In some embodiments, the suture device may include a handle located at a proximal end portion of the suture device and a suturing mechanism coupled to a distal end portion of the needle guide. The suturing mechanism may be associated with the first and second needles. The suturing mechanism may include a pivotal element capable of being moved between an insertion and removal orientation and a deployed orientation. The suturing mechanism may be located at a distal end portion of the suture device. A suturing mechanism actuator may be located on the handle, coupled to the suturing mechanism, and configured to move the suturing mechanism between the insertion and removal orientation and the deployed orientation. The suture device may include an elongated shaft coupled between the handle and the needle guide. The suturing mechanism may include a biasing member configured to return the suturing mechanism to the insertion and removal orientation. The suturing mechanism actuator may include a biasing member configured to return the suturing mechanism to the insertion and removal orientation. The suturing mechanism actuator biasing member may be a spring.

In some embodiments, the suturing mechanism may be in the insertion and removal orientation when the suturing mechanism actuator is in a first suturing mechanism actuator position. The suturing mechanism may be in the deployed orientation when the suturing mechanism actuator is in a second suturing mechanism actuator position. The suturing mechanism actuator may include a suturing mechanism actuator shaft extending though an opening in the handle and the opening in the handle may limit and guide the movement of the suturing mechanism actuator shaft. The opening in the handle may be a slot. The suturing mechanism actuator may be in the first suturing mechanism actuator position when the suturing mechanism actuator shaft is at a first end of the slot. The suturing mechanism actuator may be in the second suturing mechanism actuator position when the suturing mechanism actuator shaft is at a second end of the slot. The slot may be a “J” shaped slot. The suturing mechanism actuator may include a selector knob coupled to the suturing mechanism actuator shaft and configured to enable a user to move the suturing mechanism actuator between the first suturing mechanism actuator position and the second suturing mechanism actuator position. The suturing mechanism actuator may include a biasing member configured to maintain the suturing mechanism actuator in at least one of the first suturing mechanism actuator position or the second suturing mechanism actuator position. The suturing mechanism actuator biasing member may be a spring.

In alternative or additional aspects, the suturing mechanism may include a coupling member configured to pull the tensile member through the tissue and having first and second ends, the first end capable of being coupled to the first needle and the second end capable of being coupled to the second needle. At least one of the first or second needles may include a barbed needle tip and the coupling member may include at least one tab configured to grip the barbed needle tip. The coupling member may include a plurality of notches configured to allow the coupling member to deform. The notches may be spaced along the length of the coupling member and the spacing of the notches may at least partially define a bend radius of the coupling member. The notches may have a width and the width of the notches may at least partially define a bend radius of the coupling member. The notches may have a plurality of widths and the widths of the notches may at least partially define a plurality of bend radii of the coupling member. The notches may be configured to allow the coupling member to deform in a single plane. The coupling member may be configured to pass through the suturing mechanism in one direction. The coupling member may include at least one tab configured to prevent the coupling member from backing out of the suturing mechanism.

In some embodiments, the first needle may include a detachable needle tip coupled to the tensile member. The suture device may include at least one of a guidewire lumen or a flushing lumen extending from a proximal end portion to a distal end portion of the suture device. The suture knot may be configured to be deployed while a guidewire is in the opening. The suture device may include at least one locking tab configured to couple the suture device to an introducer sheath when at least a portion of the suture device is directed through the introducer sheath. The suture device may be part of a suture device system that includes an introducer sheath. The introducer sheath may be configured to be introduced into the opening in the tissue and the introducer sheath may be configured to be withdrawn from the opening with at least a portion of the suture device remaining in the opening. The suture device system may include a knot pusher configured to advance the suture knot along the tensile member.

An alternative suture device is provided. The alternative suture device includes a tensile member, a suture knot associated with the tensile member, and a needle guide. The needle guide includes a first needle coupled to the tensile member, a second needle, and a tensile member groove configured to accommodate the tensile member. At least a portion of the suture device is configured to be directed into an opening in a tissue. The first and second needles are configured to be directed through the tissue adjacent to the opening to direct the tensile member through the tissue. The tensile member is configured to be deployed from the tensile member groove when at least one of the first or second needles is directed through the tissue. The tensile member is configured to be pulled through the tissue when at least one of the first or second needles is retracted. The suture knot is configured to be deployed from the suture knot recess and along the tensile member and tightened to close and seal the opening.

In some embodiments, the tensile member groove may extend from a first needle side to a second needle side of the needle guide. The suture knot may be deployed when at least one of the first or second needles is retracted. The tensile member may be drawn through the suture knot when at least one of the first or second needles is retracted. The suture device may include a suture knot recess configured to accept the suture knot. The suture device may include a suture knot sleeve. The suture knot sleeve may be at least partially inside the suture knot recess. The suture knot may be wrapped around the suture knot sleeve. The suture knot sleeve may retain the suture knot until the suture knot is deployed. The suture knot may be a sliding knot. The suture knot may be a “Tennessee Slider” knot.

In alternative or additional aspects, the suture device may include a handle located at a proximal end portion of the suture device, an elongated shaft coupled between the handle and the needle guide, and a needle actuator located on the handle. The needle actuator may be coupled to at least one of the first or second needles. The needle actuator may be configured to direct at least one of the first or second needles through the tissue when the needle actuator is activated. The needle actuator may include a biasing member configured to retract at least one of the first or second needles. The needle actuator biasing member may be a spring. The needle actuator may be coupled to the first and second needles. The needle actuator may be in a first needle actuator position prior to directing the first and second needles through the tissue. The needle actuator may be configured to be moved to a second needle actuator position to direct the first and second needles through the tissue. The needle actuator may be configured to be moved to a third needle actuator position to retract the first and second needles. The needle actuator may include a plunger and a biasing member. The needle actuator may move from the first needle actuator position to the second needle actuator position when the plunger is depressed. The needle actuator biasing member may move the needle actuator from the second needle actuator position to the third needle actuator position when the plunger is released. The needle actuator biasing member may be a spring.

In alternative embodiments, the needle actuator may include a sprag. The sprag may move from a first sprag orientation to a second sprag orientation when the plunger is depressed. The sprag may allow the needle actuator to move from the second needle actuator position to the third needle actuator position when the plunger is released when the sprag is in the second sprag orientation. The sprag may be configured to rotate from the first sprag orientation to the second sprag orientation when moved by the plunger. The sprag may engage a first sprag groove when the needle actuator is in the first needle actuator position. The sprag may engage a second sprag groove when the plunger is depressed thereby allowing the needle actuator to move from the second needle actuator position to the third needle actuator position when the plunger is released.

In some embodiments, the needle actuator may include a catch. When the plunger is depressed, the plunger may move the catch, and when moved, the catch may allow the needle actuator to move from the second needle actuator position to the third needle actuator position when the plunger is released. At least a portion of the catch may be magnetic. The catch may engage a catch groove when the needle actuator is in the first needle actuator position, and the catch may engage a catch recess when the plunger is depressed thereby allowing the needle actuator to move from the second needle actuator position to the third needle actuator position when the plunger is released. The needle actuator may include a ferromagnetic portion. At least a portion of the catch may be magnetic, and the catch may be drawn into the catch recess by the ferromagnetic portion.

In alternative or additional aspects, the suture device may include a coupling member configured to pull the tensile member through the tissue and having first and second ends, the first end capable of being coupled to the first needle and the second end capable of being coupled to the second needle. At least one of the first or second needles may include a barbed needle tip and the coupling member may include at least one tab configured to grip the barbed needle tip. The coupling member may include a plurality of notches configured to allow the coupling member to deform. The notches may be spaced along the length of the coupling member and the spacing of the notches may at least partially define a bend radius of the coupling member. The notches may have a width and the width of the notches may at least partially define a bend radius of the coupling member. The notches may have a plurality of widths and the widths of the notches may at least partially define a plurality of bend radii of the coupling member.

In some embodiments, the suture device may include a handle located at a proximal end portion of the suture device, a suturing mechanism coupled to a distal end portion of the needle guide, a suturing mechanism actuator located on the handle, and an elongated shaft coupled between the handle and the needle guide. The suturing mechanism may be associated with the first and second needles. The suturing mechanism may include a pivotal element located at a distal end portion of the suture device and capable of being moved between an insertion and removal orientation and a deployed orientation. The suturing mechanism actuator may be coupled to the suturing mechanism and may be configured to move the suturing mechanism between the insertion and removal orientation and the deployed orientation. The suturing mechanism may include a biasing member configured to return the suturing mechanism to the insertion and removal orientation. The suturing mechanism actuator may include a biasing member configured to return the suturing mechanism to the insertion and removal orientation. The suturing mechanism actuator biasing member may be a spring.

In alternative or additional aspects, the suturing mechanism may be in the insertion and removal orientation when the suturing mechanism actuator is in a first suturing mechanism actuator position. The suturing mechanism may be in the deployed orientation when the suturing mechanism actuator is in a second suturing mechanism actuator position. The suture suturing mechanism actuator may include a suturing mechanism actuator shaft extending though an opening in the handle and the opening in the handle may limit and guide the movement of the suturing mechanism actuator shaft. The opening in the handle may be a slot. The suturing mechanism actuator may be in the first suturing mechanism actuator position when the suturing mechanism actuator shaft is at a first end of the slot. The suturing mechanism actuator may be in the second suturing mechanism actuator position when the suturing mechanism actuator shaft is at a second end of the slot. The slot may be a “J” shaped slot. The suturing mechanism actuator may include a selector knob coupled to the suturing mechanism actuator shaft and configured to enable a user to move the suturing mechanism actuator between the first suturing mechanism actuator position and the second suturing mechanism actuator position. The suturing mechanism actuator may include a biasing member configured to maintain the suturing mechanism actuator in at least one of the first suturing mechanism actuator position or the second suturing mechanism actuator position. The suturing mechanism actuator biasing member may be a spring.

In alternative embodiments, the suturing mechanism may include a coupling member configured to pull the tensile member through the tissue and having first and second ends, the first end capable of being coupled to the first needle and the second end capable of being coupled to the second needle. At least one of the first or second needles may include a barbed needle tip and the coupling member may include at least one tab configured to grip the barbed needle tip. The coupling member may include a plurality of notches configured to allow the coupling member to deform. The notches may be spaced along the length of the coupling member and the spacing of the notches may at least partially define a bend radius of the coupling member. The notches may have a width and the width of the notches may at least partially define a bend radius of the coupling member. The notches may have a plurality of widths and the widths of the notches may at least partially define a plurality of bend radii of the coupling member. The notches may be configured to allow the coupling member to deform in a single plane. The coupling member may be configured to pass through the suturing mechanism in one direction. The coupling member may include at least one tab configured to prevent the coupling member from backing out of the suturing mechanism.

In some embodiments, the first needle may include a detachable needle tip coupled to the tensile member. The suture device may include at least one of a guidewire lumen or a flushing lumen extending from a proximal end portion to a distal end portion of the suture device. The suture knot may be configured to be deployed while a guidewire is in the opening. The suture device may include at least one locking tab configured to couple the suture device to an introducer sheath when at least a portion of the suture device is directed through the introducer sheath. The suture device may be part of a suture device system that includes an introducer sheath. The introducer sheath may be configured to be introduced into the opening in the tissue and the introducer sheath may be configured to be withdrawn from the opening with at least a portion of the suture device remaining in the opening. The suture device system may include a knot pusher configured to advance the suture knot along the tensile member.

An alternative suture device is provided. The alternative suture device includes a tensile member, a suture knot associated with the tensile member, a needle guide, a handle located at a proximal end of the suture device, an elongated shaft coupled between the handle and the needle guide, and a needle actuator. The needle guide includes a first needle coupled to the tensile member, a second needle, and a suture knot recess configured to accept the suture knot. The needle actuator is located on the handle and is coupled to at least one of the first or second needles. At least a portion of the suture device is configured to be directed into an opening in tissue. The needle actuator is configured to direct the first and second needles through the tissue adjacent to the opening to direct the tensile member through the tissue when the needle actuator is activated. The tensile member is configured to be pulled through the tissue when at least one of the first or second needles is retracted. The suture knot is configured to be deployed from the suture knot recess and along the tensile member and tightened to close and seal the opening.

In some embodiments, the suture knot may be deployed when at least one of the first or second needles is retracted. The tensile member may be drawn through the suture knot when at least one of the first or second needles is retracted. The suture device may include a suture knot sleeve. The suture knot sleeve may be at least partially inside the suture knot recess. The suture knot may be wrapped around the suture knot sleeve. The suture knot sleeve may retain the suture knot until the suture knot is deployed. The suture knot may be a sliding knot. The suture knot may be a “Tennessee Slider” knot. The suture device may include a tensile member groove configured to accommodate the tensile member and the tensile member may be configured to be deployed from the tensile member groove when at least one of the first or second needles is directed through the tissue. The tensile member groove may extend from a first needle side to a second needle side of the needle guide.

In alternative embodiments, the needle actuator may include a biasing member configured to retract the first and second needles. The needle actuator biasing member may be a spring. The needle actuator may be in a first needle actuator position prior to directing the first and second needles through the tissue. The needle actuator may be configured to be moved to a second needle actuator position to direct the first and second needles through the tissue. The needle actuator may be configured to be moved to a third needle actuator position to retract the first and second needles. The needle actuator may include a plunger and a biasing member. The needle actuator may move from the first needle actuator position to the second needle actuator position when the plunger is depressed. The needle actuator biasing member may move the needle actuator from the second needle actuator position to the third needle actuator position when the plunger is released. The needle actuator biasing member may be a spring.

In alternative or additional aspects, the needle actuator may include a sprag. The sprag may move from a first sprag orientation to a second sprag orientation when the plunger is depressed. The sprag may allow the needle actuator to move from the second needle actuator position to the third needle actuator position when the plunger is released when the sprag is in the second sprag orientation. The sprag may be configured to rotate from the first sprag orientation to the second sprag orientation when moved by the plunger. The sprag may engage a first sprag groove when the needle actuator is in the first needle actuator position. The sprag may engage a second sprag groove when the plunger is depressed thereby allowing the needle actuator to move from the second needle actuator position to the third needle actuator position when the plunger is released.

In some embodiments, the needle actuator may include a catch. When the plunger is depressed, the plunger may move the catch, and when moved, the catch may allow the needle actuator to move from the second needle actuator position to the third needle actuator position when the plunger is released. At least a portion of the catch may be magnetic. The catch may engage a catch groove when the needle actuator is in the first needle actuator position, and the catch may engage a catch recess when the plunger is depressed thereby allowing the needle actuator to move from the second needle actuator position to the third needle actuator position when the plunger is released. The needle actuator may include a ferromagnetic portion. At least a portion of the catch may be magnetic, and the catch may be drawn into the catch recess by the ferromagnetic portion.

In alternate or additional aspects, the suture device may include a coupling member configured to pull the tensile member through the tissue and having first and second ends, the first end capable of being coupled to the first needle and the second end capable of being coupled to the second needle. At least one of the first or second needles may include a barbed needle tip and the coupling member may include at least one tab configured to grip the barbed needle tip. The coupling member may include a plurality of notches configured to allow the coupling member to deform. The notches may be spaced along the length of the coupling member and the spacing of the notches may at least partially define a bend radius of the coupling member. The notches may have a width and the width of the notches may at least partially define a bend radius of the coupling member. The notches may have a plurality of widths and the widths of the notches may at least partially define a plurality of bend radii of the coupling member. The notches may be configured to allow the coupling member to deform in a single plane.

In some embodiments, the suture device may include a suturing mechanism and a suturing mechanism actuator. The suturing mechanism may be coupled to a distal end portion of the needle guide. The suturing mechanism may be associated with the first and second needles. The suturing mechanism may include a pivotal element located at a distal end portion of the suture device and capable of being moved between an insertion and removal orientation and a deployed orientation. The suturing mechanism actuator may be located on the handle. The suturing mechanism actuator may be coupled to the suturing mechanism and configured to move the suturing mechanism between the insertion and removal orientation and the deployed orientation. The suturing mechanism may include a biasing member configured to return the suturing mechanism to the insertion and removal orientation. The suturing mechanism actuator may include a biasing member configured to return the suturing mechanism to the insertion and removal orientation. The suturing mechanism actuator biasing member may be a spring.

In alternative embodiments, the suturing mechanism may be in the insertion and removal orientation when the suturing mechanism actuator is in a first suturing mechanism actuator position. The suturing mechanism may be in the deployed orientation when the suturing mechanism actuator is in a second suturing mechanism actuator position. The suture suturing mechanism actuator may include a suturing mechanism actuator shaft extending though an opening in the handle and the opening in the handle may limit and guide the movement of the suturing mechanism actuator shaft. The opening in the handle may be a slot. The suturing mechanism actuator may be in the first suturing mechanism actuator position when the suturing mechanism actuator shaft is at a first end of the slot. The suturing mechanism actuator may be in the second suturing mechanism actuator position when the suturing mechanism actuator shaft is at a second end of the slot. The slot may be a “J” shaped slot. The suturing mechanism actuator may include a selector knob coupled to the suturing mechanism actuator shaft and configured to enable a user to move the suturing mechanism actuator between the first suturing mechanism actuator position and the second suturing mechanism actuator position. The suturing mechanism actuator may include a biasing member configured to maintain the suturing mechanism actuator in at least one of the first suturing mechanism actuator position or the second suturing mechanism actuator position. The suturing mechanism actuator biasing member may be a spring.

In alternative or additional aspects, the suturing mechanism may include a coupling member configured to pull the tensile member through the tissue and having first and second ends, the first end capable of being coupled to the first needle and the second end capable of being coupled to the second needle. At least one of the first or second needles may include a barbed needle tip and the coupling member may include at least one tab configured to grip the barbed needle tip. The coupling member may include a plurality of notches configured to allow the coupling member to deform. The notches may be spaced along the length of the coupling member and the spacing of the notches may at least partially define a bend radius of the coupling member. The notches may have a width and the width of the notches may at least partially define a bend radius of the coupling member. The notches may have a plurality of widths and the widths of the notches may at least partially define a plurality of bend radii of the coupling member. The notches may be configured to allow the coupling member to deform in a single plane. The coupling member may be configured to pass through the suturing mechanism in one direction. The coupling member may include at least one tab configured to prevent the coupling member from backing out of the suturing mechanism.

In some embodiments, the first needle may include a detachable needle tip coupled to the tensile member. The suture device may include at least one of a guidewire lumen or a flushing lumen extending from a proximal end portion to a distal end portion of the suture device. The suture knot may be configured to be deployed while a guidewire is in the opening. The suture device may include at least one locking tab configured to couple the suture device to an introducer sheath when at least a portion of the suture device is directed through the introducer sheath. The suture device may be part of a suture device system that includes an introducer sheath. The introducer sheath may be configured to be introduced into the opening in the tissue and the introducer sheath may be configured to be withdrawn from the opening with at least a portion of the suture device remaining in the opening. The suture device system may include a knot pusher configured to advance the suture knot along the tensile member.

An alternative suture device is provided. The alternative suture device includes a tensile member, a suture knot associated with the tensile member, a needle guide, a handle located at a proximal end of the suture device, an elongated shaft coupled between the handle and the needle guide, and a needle actuator. The needle guide includes a first needle coupled to the tensile member, a second needle, and a tensile member groove configured to accommodate the tensile member. The needle actuator is located on the handle and coupled to at least one of the first or second needles. At least a portion of the suture device is configured to be directed into an opening in tissue. The needle actuator is configured to direct the first and second needles through the tissue adjacent to the opening to direct the tensile member through the tissue when the needle actuator is activated. The tensile member is configured to be deployed from the tensile member groove when at least one of the first or second needles is directed through the tissue. The tensile member is configured to be pulled through the tissue when at least one of the first or second needles is retracted. The suture knot is configured to be deployed along the tensile member and tightened to close and seal the opening.

In some embodiments, the tensile member groove may extend from a first needle side to a second needle side of the needle guide. The suture knot may be deployed when at least one of the first or second needles is retracted. The tensile member may be drawn through the suture knot when at least one the first or second needles is retracted. The suture device may include a suture knot recess configured to accept the suture knot. The suture device may include a suture knot sleeve. The suture knot sleeve may be at least partially inside the suture knot recess. The suture knot may be wrapped around the suture knot sleeve. The suture knot sleeve may retain the suture knot until the suture knot is deployed. The suture knot may be a sliding knot. The suture knot may be a “Tennessee Slider” knot.

In alternative embodiments, the needle actuator may include a biasing member configured to retract at least one of the first or second needles. The needle actuator biasing member may be a spring. The needle actuator may be in a first needle actuator position prior to directing the first and second needles through the tissue. The needle actuator may be configured to be moved to a second needle actuator position to direct the first and second needles through the tissue. The needle actuator may be configured to be moved to a third needle actuator position to retract the first and second needles.

In alternative or additional aspects, the needle actuator may include a plunger and a biasing member. The needle actuator may move from the first needle actuator position to the second needle actuator position when the plunger is depressed. The needle actuator biasing member may move the needle actuator from the second needle actuator position to the third needle actuator position when the plunger is released. The needle actuator biasing member may be a spring.

In alternative or additional aspects, the needle actuator may include a sprag. The sprag may move from a first sprag orientation to a second sprag orientation when the plunger is depressed. The sprag may allow the needle actuator to move from the second needle actuator position to the third needle actuator position when the plunger is released when the sprag is in the second sprag orientation. The sprag may be configured to rotate from the first sprag orientation to the second sprag orientation when moved by the plunger. The sprag may engage a first sprag groove when the needle actuator is in the first needle actuator position. The sprag may engage a second sprag groove when the plunger is depressed thereby allowing the needle actuator to move from the second needle actuator position to the third needle actuator position when the plunger is released.

In some embodiments, the needle actuator may include a catch. When the plunger is depressed, the plunger may move the catch, and when moved, the catch may allow the needle actuator to move from the second needle actuator position to the third needle actuator position when the plunger is released. At least a portion of the catch may be magnetic. The catch may engage a catch groove when the needle actuator is in the first needle actuator position, and the catch may engage a catch recess when the plunger is depressed thereby allowing the needle actuator to move from the second needle actuator position to the third needle actuator position when the plunger is released. The needle actuator may include a ferromagnetic portion. At least a portion of the catch may be magnetic, and the catch may be drawn into the catch recess by the ferromagnetic portion.

In alternate or additional aspects, the suture device may include a coupling member configured to pull the tensile member through the tissue and having first and second ends, the first end capable of being coupled to the first needle and the second end capable of being coupled to the second needle. At least one of the first or second needles may include a barbed needle tip and the coupling member may include at least one tab configured to grip the barbed needle tip. The coupling member may include a plurality of notches configured to allow the coupling member to deform. The notches may be spaced along the length of the coupling member and the spacing of the notches may at least partially define a bend radius of the coupling member. The notches may have a width and the width of the notches may at least partially define a bend radius of the coupling member. The notches may have a plurality of widths and the widths of the notches may at least partially define a plurality of bend radii of the coupling member. The notches may be configured to allow the coupling member to deform in a single plane.

In some embodiments, the suture device may include a suturing mechanism and a suturing mechanism actuator. The suturing mechanism may be coupled to a distal end portion of the needle guide. The suturing mechanism may be associated with the first and second needles. The suturing mechanism may include a pivotal element located at a distal end portion of the suture device and capable of being moved between an insertion and removal orientation and a deployed orientation. The suturing mechanism actuator may be located on the handle. The suturing mechanism actuator may be coupled to the suturing mechanism and configured to move the suturing mechanism between the insertion and removal orientation and the deployed orientation. The suturing mechanism may include a biasing member configured to return the suturing mechanism to the insertion and removal orientation. The suturing mechanism actuator may include a biasing member configured to return the suturing mechanism to the insertion and removal orientation. The suturing mechanism actuator biasing member may be a spring.

The suturing mechanism may be in the insertion and removal orientation when the suturing mechanism actuator is in a first suturing mechanism actuator position. The suturing mechanism may be in the deployed orientation when the suturing mechanism actuator is in a second suturing mechanism actuator position. The suture suturing mechanism actuator may include a suturing mechanism actuator shaft extending though an opening in the handle and the opening in the handle may limit and guide the movement of the suturing mechanism actuator shaft. The opening in the handle may be a slot. The suturing mechanism actuator may be in the first suturing mechanism actuator position when the suturing mechanism actuator shaft is at a first end of the slot. The suturing mechanism actuator may be in the second suturing mechanism actuator position when the suturing mechanism actuator shaft is at a second end of the slot. The slot may be a “J” shaped slot. The suturing mechanism actuator may include a selector knob coupled to the suturing mechanism actuator shaft and configured to enable a user to move the suturing mechanism actuator between the first suturing mechanism actuator position and the second suturing mechanism actuator position. The suturing mechanism actuator may include a biasing member configured to maintain the suturing mechanism actuator in at least one of the first suturing mechanism actuator position or the second suturing mechanism actuator position. The suturing mechanism actuator biasing member may be a spring.

In alternate or additional aspects, the suturing mechanism may include a coupling member configured to pull the tensile member through the tissue and having first and second ends, the first end capable of being coupled to the first needle and the second end capable of being coupled to the second needle. At least one of the first or second needles may include a barbed needle tip and the coupling member may include at least one tab configured to grip the barbed needle tip. The coupling member may include a plurality of notches configured to allow the coupling member to deform. The notches may be spaced along the length of the coupling member and the spacing of the notches may at least partially define a bend radius of the coupling member. The notches may have a width and the width of the notches may at least partially define a bend radius of the coupling member. The notches may have a plurality of widths and the widths of the notches may at least partially define a plurality of bend radii of the coupling member. The notches may be configured to allow the coupling member to deform in a single plane. The coupling member may be configured to pass through the suturing mechanism in one direction. The coupling member may include at least one tab configured to prevent the coupling member from backing out of the suturing mechanism.

In some embodiments, the first needle may include a detachable needle tip coupled to the tensile member. The suture device may include at least one of a guidewire lumen or a flushing lumen extending from a proximal end portion to a distal end portion of the suture device. The suture knot may be configured to be deployed while a guidewire is in the opening. The suture device may include at least one locking tab configured to couple the suture device to an introducer sheath when at least a portion of the suture device is directed through the introducer sheath. The suture device may be part of a suture device system that includes an introducer sheath. The introducer sheath may be configured to be introduced into the opening in the tissue and the introducer sheath may be configured to be withdrawn from the opening with at least a portion of the suture device remaining in the opening. The suture device system may include a knot pusher configured to advance the suture knot along the tensile member.

An alternative suture device is provided. The alternative suture device includes a tensile member, a suture knot associated with the tensile member; and a needle guide. The needle guide includes a first needle coupled to the tensile member, a second needle, and a suture knot sleeve configured to accept the suture knot. At least a portion of the suture device is configured to be directed into an opening in a tissue. The first and second needles are configured to be directed through the tissue adjacent to the opening to direct the tensile member through the tissue. The tensile member is configured to be pulled through the tissue when at least one of the first or second needles is retracted. The suture knot is wrapped around the suture knot sleeve. The suture knot is configured to be deployed from the suture knot sleeve and along the tensile member and tightened to close and seal the opening.

In some embodiments, the suture knot may be deployed when at least one of the first or second needles is retracted. The tensile member may be drawn through the suture knot when at least one of the first or second needles is retracted. The suture device may include a suture knot recess configured to accept the suture knot. The suture knot sleeve may be at least partially inside the suture knot recess. The needle guide may include a tensile member groove configured to accommodate the tensile member. The tensile member may be configured to be deployed from the tensile member groove when at least one of the first or second needles is directed through the tissue. The tensile member groove may extend from a first needle side to a second needle side of the needle guide. The suture knot may be a sliding knot. The suture knot may be a “Tennessee Slider” knot.

In alternative embodiments, the suture device may include a handle located at a proximal end portion of the suture device, an elongated shaft coupled between the handle and the needle guide, and a needle actuator located on the handle. The needle actuator may be coupled to at least one of the first or second needles, and configured to direct at least one of the first or second needles through the tissue when the needle actuator is activated. The needle actuator may include a biasing member configured to retract at least one of the first or second needles. The needle actuator biasing member may be a spring.

In alternative or additional aspects, the needle actuator may be coupled to the first and second needles. The needle actuator may be in a first needle actuator position prior to directing the first and second needles through the tissue. The needle actuator may be configured to be moved to a second needle actuator position to direct the first and second needles through the tissue. The needle actuator may be configured to be moved to a third needle actuator position to retract the first and second needles.

In alternative or additional aspects, the needle actuator may include a plunger and a biasing member. The needle actuator may move from the first needle actuator position to the second needle actuator position when the plunger is depressed. The needle actuator biasing member may move the needle actuator from the second needle actuator position to the third needle actuator position when the plunger is released. The needle actuator biasing member may be a spring. The needle actuator biasing member may be a spring. The needle actuator may include a sprag. The sprag may move from a first sprag orientation to a second sprag orientation when the plunger is depressed. The sprag may allow the needle actuator to move from the second needle actuator position to the third needle actuator position when the plunger is released and the sprag is in the second sprag orientation. The sprag may be configured to rotate from the first sprag orientation to the second sprag orientation. The sprag may engage a first sprag groove when the needle actuator is in the first needle actuator position. The sprag may engage a second sprag groove when the plunger is depressed thereby allowing the needle actuator to move from the second needle actuator position to the third needle actuator position when the plunger is released.

In some embodiments, the needle actuator may include a catch. When the plunger is depressed, the plunger may move the catch, and when moved, the catch may allow the needle actuator to move from the second needle actuator position to the third needle actuator position when the plunger is released. At least a portion of the catch may be magnetic. The catch may engage a catch groove when the needle actuator is in the first needle actuator position, and the catch may engage a catch recess when the plunger is depressed thereby allowing the needle actuator to move from the second needle actuator position to the third needle actuator position when the plunger is released. The needle actuator may include a ferromagnetic portion. At least a portion of the catch may be magnetic, and the catch may be drawn into the catch recess by the ferromagnetic portion.

In alternative or additional aspects, the suture device may include a coupling member configured to pull the tensile member through the tissue and having first and second ends, the first end capable of being coupled to the first needle and the second end capable of being coupled to the second needle. At least one of the first or second needles may include a barbed needle tip and the coupling member may include at least one tab configured to grip the barbed needle tip. The coupling member may include a plurality of notches configured to allow the coupling member to deform. The notches may be spaced along the length of the coupling member and the spacing of the notches may at least partially define a bend radius of the coupling member. The notches may have a width and the width of the notches may at least partially define a bend radius of the coupling member. The notches may have a plurality of widths and the widths of the notches may at least partially define a plurality of bend radii of the coupling member. The notches may be configured to allow the coupling member to deform in a single plane.

In some embodiments, the suture device may include a handle located at a proximal end portion of the suture device and a suturing mechanism coupled to a distal end portion of the needle guide. The suturing mechanism may be associated with the first and second needles. The suturing mechanism may include a pivotal element capable of being moved between an insertion and removal orientation and a deployed orientation. The suturing mechanism may be located at a distal end portion of the suture device. A suturing mechanism actuator may be located on the handle, coupled to the suturing mechanism, and configured to move the suturing mechanism between the insertion and removal orientation and the deployed orientation. The suture device may include an elongated shaft coupled between the handle and the needle guide. The suturing mechanism may include a biasing member configured to return the suturing mechanism to the insertion and removal orientation. The suturing mechanism actuator may include a biasing member configured to return the suturing mechanism to the insertion and removal orientation. The suturing mechanism actuator biasing member may be a spring.

In some embodiments, the suturing mechanism may be in the insertion and removal orientation when the suturing mechanism actuator is in a first suturing mechanism actuator position. The suturing mechanism may be in the deployed orientation when the suturing mechanism actuator is in a second suturing mechanism actuator position. The suturing mechanism actuator may include a suturing mechanism actuator shaft extending though an opening in the handle and the opening in the handle may limit and guide the movement of the suturing mechanism actuator shaft. The opening in the handle may be a slot. The suturing mechanism actuator may be in the first suturing mechanism actuator position when the suturing mechanism actuator shaft is at a first end of the slot. The suturing mechanism actuator may be in the second suturing mechanism actuator position when the suturing mechanism actuator shaft is at a second end of the slot. The slot may be a “J” shaped slot. The suturing mechanism actuator may include a selector knob coupled to the suturing mechanism actuator shaft and configured to enable a user to move the suturing mechanism actuator between the first suturing mechanism actuator position and the second suturing mechanism actuator position. The suturing mechanism actuator may include a biasing member configured to maintain the suturing mechanism actuator in at least one of the first suturing mechanism actuator position or the second suturing mechanism actuator position. The suturing mechanism actuator biasing member may be a spring.

In alternate or additional aspects, the suturing mechanism may include a coupling member configured to pull the tensile member through the tissue and having first and second ends, the first end capable of being coupled to the first needle and the second end capable of being coupled to the second needle. At least one of the first or second needles may include a barbed needle tip and the coupling member may include at least one tab configured to grip the barbed needle tip. The coupling member may include a plurality of notches configured to allow the coupling member to deform. The notches may be spaced along the length of the coupling member and the spacing of the notches may at least partially define a bend radius of the coupling member. The notches may have a width and the width of the notches may at least partially define a bend radius of the coupling member. The notches may have a plurality of widths and the widths of the notches may at least partially define a plurality of bend radii of the coupling member. The notches may be configured to allow the coupling member to deform in a single plane. The coupling member may be configured to pass through the suturing mechanism in one direction. The coupling member may include at least one tab configured to prevent the coupling member from backing out of the suturing mechanism.

In some embodiments, the first needle may include a detachable needle tip coupled to the tensile member. The suture device may include at least one of a guidewire lumen or a flushing lumen extending from a proximal end portion to a distal end portion of the suture device. The suture knot may be configured to be deployed while a guidewire is in the opening. The suture device may include at least one locking tab configured to couple the suture device to an introducer sheath when at least a portion of the suture device is directed through the introducer sheath. The suture device may be part of a suture device system that includes an introducer sheath. The introducer sheath may be configured to be introduced into the opening in the tissue and the introducer sheath may be configured to be withdrawn from the opening with at least a portion of the suture device remaining in the opening. The suture device system may include a knot pusher configured to advance the suture knot along the tensile member.

An alternative suture device is provided. The alternative suture device includes a tensile member, a suture knot associated with the tensile member, a needle guide, a handle located at a proximal end of the suture device, an elongated shaft coupled between the handle and the needle guide, and a needle actuator. The needle guide includes a first needle coupled to the tensile member, a second needle, and a suture knot sleeve configured to accept the suture knot. The needle actuator is located on the handle and is coupled to at least one of the first or second needles. At least a portion of the suture device is configured to be directed into an opening in tissue. The needle actuator is configured to direct the first and second needles through the tissue adjacent to the opening to direct the tensile member through the tissue when the needle actuator is activated. The tensile member is configured to be pulled through the tissue when at least one of the first or second needles is retracted. The suture knot is wrapped around the suture knot sleeve. The suture knot is configured to be deployed from the suture knot sleeve and along the tensile member and tightened to close and seal the opening.

In some embodiments, the suture knot may be deployed when at least one of the first or second needles is retracted. The tensile member may be drawn through the suture knot when at least one of the first or second needles is retracted. The suture device may include a suture knot recess configured to accept the suture knot. The suture knot sleeve may be at least partially inside the suture knot recess. The needle guide may include a tensile member groove configured to accommodate the tensile member. The tensile member may be configured to be deployed from the tensile member groove when at least one of the first or second needles is directed through the tissue. The tensile member groove may extend from a first needle side to a second needle side of the needle guide. The suture knot may be a sliding knot. The suture knot may be a “Tennessee Slider” knot.

In alternative embodiments, the needle actuator may include a biasing member configured to retract at least one of the first or second needles. The needle actuator biasing member may be a spring. The needle actuator may be in a first needle actuator position prior to directing the first and second needles through the tissue. The needle actuator may be configured to be moved to a second needle actuator position to direct the first and second needles through the tissue. The needle actuator may be configured to be moved to a third needle actuator position to retract the first and second needles.

In alternative or additional aspects, the needle actuator may include a plunger and a biasing member. The needle actuator may move from the first needle actuator position to the second needle actuator position when the plunger is depressed. The needle actuator biasing member may move the needle actuator from the second needle actuator position to the third needle actuator position when the plunger is released. The needle actuator biasing member may be a spring. The needle actuator biasing member may be a spring. The needle actuator may include a sprag. The sprag may move from a first sprag orientation to a second sprag orientation when the plunger is depressed. The sprag may allow the needle actuator to move from the second needle actuator position to the third needle actuator position when the plunger is released and the sprag is in the second sprag orientation. The sprag may be configured to rotate from the first sprag orientation to the second sprag orientation. The sprag may engage a first sprag groove when the needle actuator is in the first needle actuator position. The sprag may engage a second sprag groove when the plunger is depressed thereby allowing the needle actuator to move from the second needle actuator position to the third needle actuator position when the plunger is released.

In some embodiments, the needle actuator may include a catch. When the plunger is depressed, the plunger may move the catch, and when moved, the catch may allow the needle actuator to move from the second needle actuator position to the third needle actuator position when the plunger is released. At least a portion of the catch may be magnetic. The catch may engage a catch groove when the needle actuator is in the first needle actuator position, and the catch may engage a catch recess when the plunger is depressed thereby allowing the needle actuator to move from the second needle actuator position to the third needle actuator position when the plunger is released. The needle actuator may include a ferromagnetic portion. At least a portion of the catch may be magnetic, and the catch may be drawn into the catch recess by the ferromagnetic portion.

In alternative or additional aspects, the suture device may include a coupling member configured to pull the tensile member through the tissue and having first and second ends, the first end capable of being coupled to the first needle and the second end capable of being coupled to the second needle. At least one of the first or second needles may include a barbed needle tip and the coupling member may include at least one tab configured to grip the barbed needle tip. The coupling member may include a plurality of notches configured to allow the coupling member to deform. The notches may be spaced along the length of the coupling member and the spacing of the notches may at least partially define a bend radius of the coupling member. The notches may have a width and the width of the notches may at least partially define a bend radius of the coupling member. The notches may have a plurality of widths and the widths of the notches may at least partially define a plurality of bend radii of the coupling member. The notches may be configured to allow the coupling member to deform in a single plane.

In alternative embodiments, the suture device may include a suturing mechanism coupled to a distal end portion of the needle guide and associated with the first and second needles and a suturing mechanism actuator located on the handle. The suturing mechanism may include a pivotal element capable of being moved between an insertion and removal orientation and a deployed orientation. The pivotal element may be located at a distal end portion of the suture device. The suturing mechanism actuator may be coupled to the suturing mechanism and configured to move the suturing mechanism between the insertion and removal orientation and the deployed orientation. The suturing mechanism may include a biasing member configured to return the suturing mechanism to the insertion and removal orientation. The suturing mechanism actuator may include a biasing member configured to return the suturing mechanism to the insertion and removal orientation. The suturing mechanism actuator biasing member may be a spring.

In some embodiments, the suturing mechanism may be in the insertion and removal orientation when the suturing mechanism actuator is in a first suturing mechanism actuator position. The suturing mechanism may be in the deployed orientation when the suturing mechanism actuator is in a second suturing mechanism actuator position. The suturing mechanism actuator may include a suturing mechanism actuator shaft extending though an opening in the handle and the opening in the handle may limit and guide the movement of the suturing mechanism actuator shaft. The opening in the handle may be a slot. The suturing mechanism actuator may be in the first suturing mechanism actuator position when the suturing mechanism actuator shaft is at a first end of the slot. The suturing mechanism actuator may be in the second suturing mechanism actuator position when the suturing mechanism actuator shaft is at a second end of the slot. The slot may be a “J” shaped slot. The suturing mechanism actuator may include a selector knob coupled to the suturing mechanism actuator shaft and configured to enable a user to move the suturing mechanism actuator between the first suturing mechanism actuator position and the second suturing mechanism actuator position. The suturing mechanism actuator may include a biasing member configured to maintain the suturing mechanism actuator in at least one of the first suturing mechanism actuator position or the second suturing mechanism actuator position. The suturing mechanism actuator biasing member may be a spring.

In alternate or additional aspects, the suturing mechanism may include a coupling member configured to pull the tensile member through the tissue and having first and second ends, the first end capable of being coupled to the first needle and the second end capable of being coupled to the second needle. At least one of the first or second needles may include a barbed needle tip and the coupling member may include at least one tab configured to grip the barbed needle tip. The coupling member may include a plurality of notches configured to allow the coupling member to deform. The notches may be spaced along the length of the coupling member and the spacing of the notches may at least partially define a bend radius of the coupling member. The notches may have a width and the width of the notches may at least partially define a bend radius of the coupling member. The notches may have a plurality of widths and the widths of the notches may at least partially define a plurality of bend radii of the coupling member. The notches may be configured to allow the coupling member to deform in a single plane. The coupling member may be configured to pass through the suturing mechanism in one direction. The coupling member may include at least one tab configured to prevent the coupling member from backing out of the suturing mechanism.

In some embodiments, the first needle may include a detachable needle tip coupled to the tensile member. The suture device may include at least one of a guidewire lumen or a flushing lumen extending from a proximal end portion to a distal end portion of the suture device. The suture knot may be configured to be deployed while a guidewire is in the opening. The suture device may include at least one locking tab configured to couple the suture device to an introducer sheath when at least a portion of the suture device is directed through the introducer sheath. The suture device may be part of a suture device system that includes an introducer sheath. The introducer sheath may be configured to be introduced into the opening in the tissue and the introducer sheath may be configured to be withdrawn from the opening with at least a portion of the suture device remaining in the opening. The suture device system may include a knot pusher configured to advance the suture knot along the tensile member.

A method of applying a suture to close and seal an opening in tissue is provided. The method of applying a suture to close and seal an opening in tissue includes directing at least one needle through the tissue adjacent to the opening, directing a tensile member through the tissue adjacent to the opening, directing at least a portion of the at least one needle through a suture knot, deploying the suture knot, and tightening the suture knot to close and seal the opening.

In some embodiments, the method may include directing the tensile member through the suture knot. The method may include deploying the suture knot from a knot recess. The method may include deploying the suture knot from a sleeve. The method may include directing at least a portion of the at least one needle through the sleeve. The method may include directing at least a portion of the tensile member through the sleeve. The method may include deploying the suture knot onto the tensile member. The suture knot may be a pre-tied knot. The method may include directing at least a portion of a suturing mechanism with a pivotal element in an insertion and removal orientation through the opening in the tissue, moving the pivotal element of the suturing mechanism into a deployed orientation, returning the pivotal element to the insertion and removal orientation and withdrawing the suturing mechanism from the opening.

In alternative embodiments, the method may include directing an introducer sheath into the opening in the tissue, directing at least a portion of the suturing mechanism through the introducer sheath, and withdrawing the introducer sheath from the opening with at least a portion of the suturing mechanism remaining in the opening. The method may include coupling the suturing mechanism to a proximal end of the introducer sheath. The suturing mechanism may include a coupling member having first and second ends. The suturing mechanism may include a first needle and a second needle. The method may include coupling the first needle to the first end of the coupling member, coupling the second needle to the second end of the coupling member, retracting the first and second needles, and pulling the tensile member through the tissue with the coupling member. The method may include directing a guidewire through the opening. The method may include deploying the suture knot while the guidewire is in the opening. The method may include withdrawing the guidewire from the opening.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination within and between the various embodiments. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept.

### BRIEF DESCRIPTION OF DRAWINGS

FIG. 1 is a perspective view of an illustrative suturing device and introducer sheath.

FIGS. 2 and 3 are perspective views of the illustrative suturing device of FIG. 1.

FIG. 4 is an exploded view of the illustrative suturing device of FIGS. 1-3.

FIGS. 5A and 5B are top views of a portion of the illustrative suturing device of FIGS. 1-3.

FIGS. 6A, 6C, and 6E are side views of the illustrative suturing device of FIGS. 1-3 showing at least some of the external features of the handle.

FIGS. 6B, 6D, and 6F are sectional side views of the illustrative suturing device of FIGS. 1-3 showing at least some of the components of the handle.

FIGS. 7A, 7B, and 7C are sectional views of a portion of the handle of the illustrative suturing device of FIGS. 1-3.

FIGS. 8A, 8B, and 8C are perspective views of the suturing mechanism of the illustrative suturing device of FIGS. 1-3.

FIGS. 9A and 9B are sectional side views of the suturing mechanism of the illustrative suturing device of FIGS. 1-3.

FIGS. 10A and 10B are perspective views of the suturing mechanism of the illustrative suturing device of FIGS. 1-3. with portions of the suturing mechanism hidden.

FIG. 11A is a side view of an illustrative coupling member.

FIG. 11B is an end view of the illustrative coupling member of FIG. 11A.

FIG. 11C is a side view of an alternative illustrative coupling member.

FIGS. 12A through 12M progressively illustrate the use of the illustrative suturing device of FIGS. 1-3, and particularly, the suturing mechanism at the distal end of the suturing device within a blood vessel.

FIG. 12N is a perspective view of an illustrative knot pusher.

FIGS. 13 and 14 are perspective views of an alternative illustrative suturing device.

FIG. 15 is an exploded view of the illustrative suturing device of FIGS. 13 and 14.

FIGS. 16A, 16C, and 16E are side views of the illustrative suturing device of FIGS. 13 and 14 showing at least some of the external features of the handle.

FIGS. 16B, 16D, and 16F are sectional side views of the illustrative suturing device of FIGS. 13 and 14 showing at least some of the components of the handle.

FIGS. 17A, 17B, and 17C are sectional views of a portion of the handle of the illustrative suturing device of FIGS. 13 and 14.

FIGS. 18A and 18B are perspective views of an alternative illustrative suturing mechanism.

FIGS. 19A and 19B are sectional views of the illustrative suturing mechanism of FIGS. 18A and 18B.

FIGS. 20A through 20M progressively illustrate the use of the illustrative suturing mechanism of FIGS. 18A and 18B and, particularly, the suturing mechanism within a blood vessel.

FIGS. 21 and 22 are perspective views of an alternative illustrative suturing device.

FIG. 23 is an exploded view of the illustrative suturing device of FIGS. 21 and 22.

FIG. 23A is an exploded view of a portion of the handle of the illustrative suturing device of FIGS. 21 and 22.

FIG. 23B is an exploded view of an alternative portion of the handle of the illustrative suturing device of FIGS. 21 and 22.

FIGS. 24A, 24D, and 24G are side views of the illustrative suturing device of FIGS. 21 and 22 showing at least some of the external features of the handle.

FIGS. 24B, 24E, and 24H are sectional side views of the illustrative suturing device of FIGS. 21 and 22 showing at least some of the components of the handle.

FIGS. 24C, 24F, and 24I are sectional top views a portion of the handle of the illustrative suturing device of FIGS. 21 and 22 showing at least some of the components of the handle.

### DETAILED DESCRIPTION

The present disclosure generally relates to devices, systems and methods for sealing punctures or other openings in blood vessels and other tissues. A device and/or system that can directly access and percutaneously seal an opening in the common carotid artery made during the direct access of the common carotid artery for neurovascular therapy is a specific example of the disclosure. The device disclosed herein generally allows for a single-suture closure of a blood vessel, such as the common carotid artery or another vessel, via a minimally invasive percutaneous approach. As used herein, the term “vessel”, “blood vessel”, “arteries”, “veins”, and similar forms of these terms mean any component of the circulatory system that transports blood throughout the human body. The devices, systems, and methods described herein may be used during other therapies or surgical procedures as well.

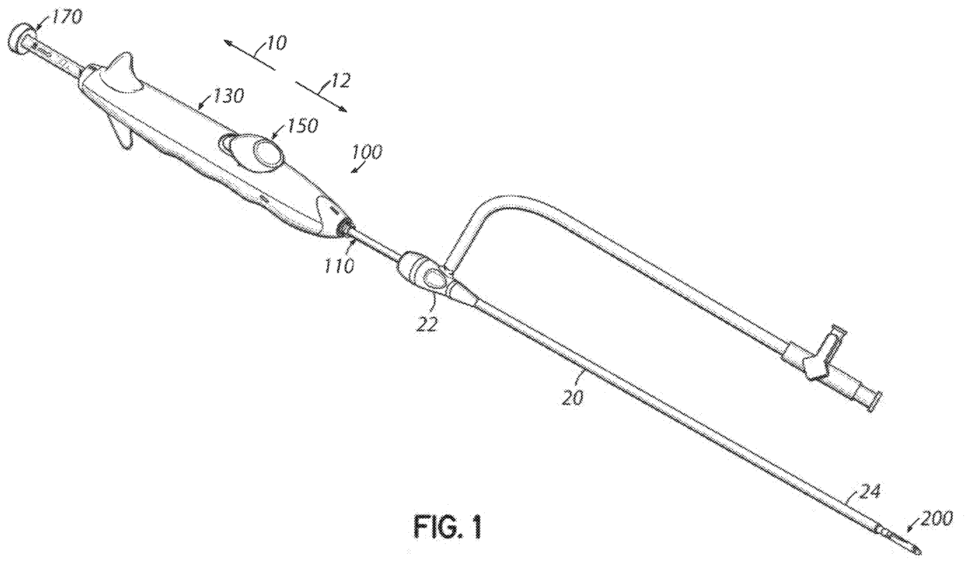

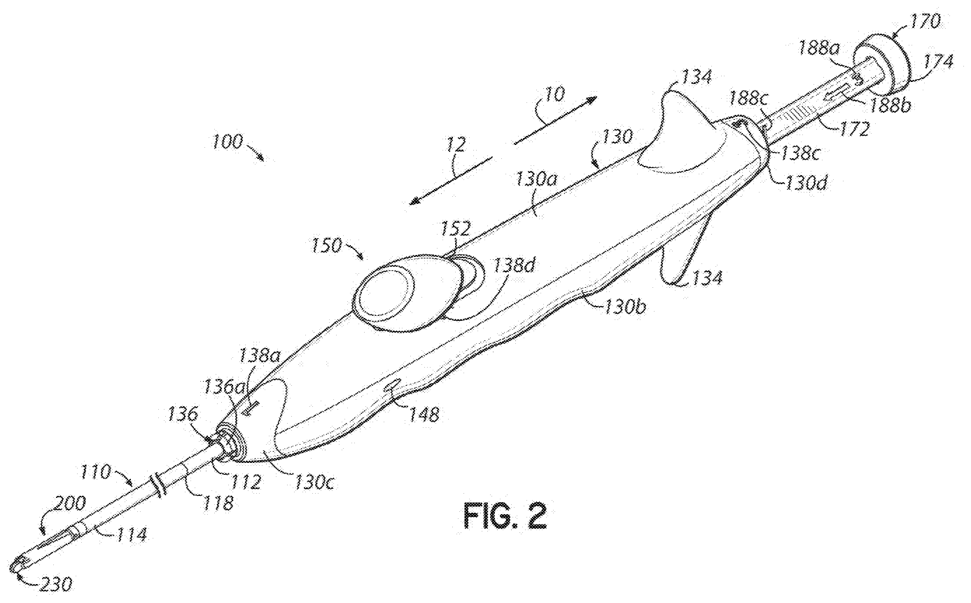

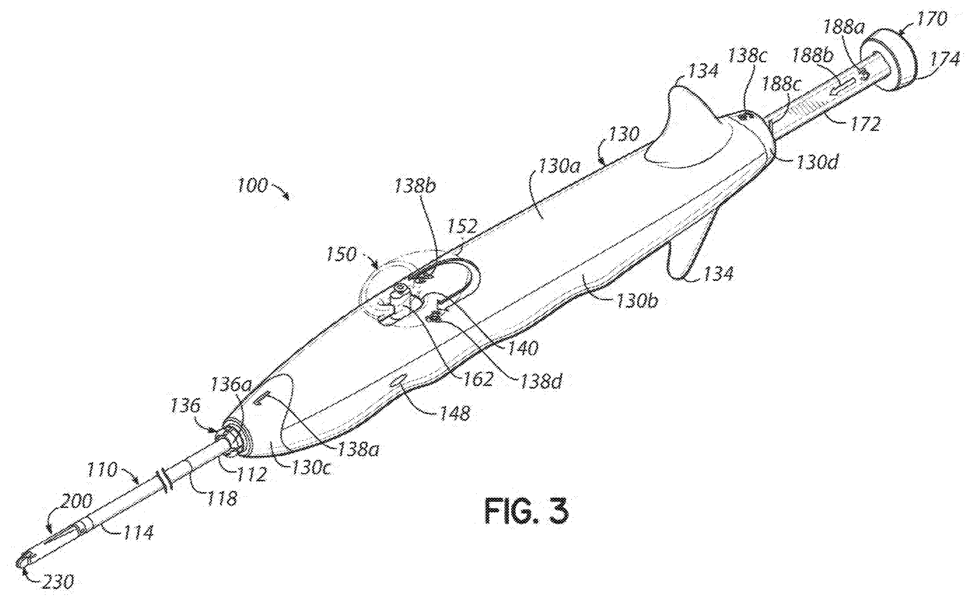

FIG. 1 is a perspective view of an illustrative suturing device 100 and introducer sheath 20, and FIGS. 2 and 3 are perspective views of the illustrative suturing device 100. The suturing device 100 includes an elongated shaft 110, a handle 130 coupled to a proximal end portion 112 of the shaft 110 and a suturing mechanism 200 coupled to the distal end portion 114 of the shaft 110. As used herein to describe various embodiments from the perspective of a user of a suturing device, “proximal” may refer to a direction generally towards the user of the device, while “distal” may refer to a direction generally away from the user of the device. For reference arrow 10 points generally in the proximal direction and arrow 12 points generally in the distal direction.

As used herein, “suture”, “suturing” and similar forms of these terms mean any flexible tensile member regardless of form or material and suitable for approximating tissue. As used herein, “tensile member” may be a mono-filament suture, a multi-filament suture, a metallic suture, or any other suitable tensile member.

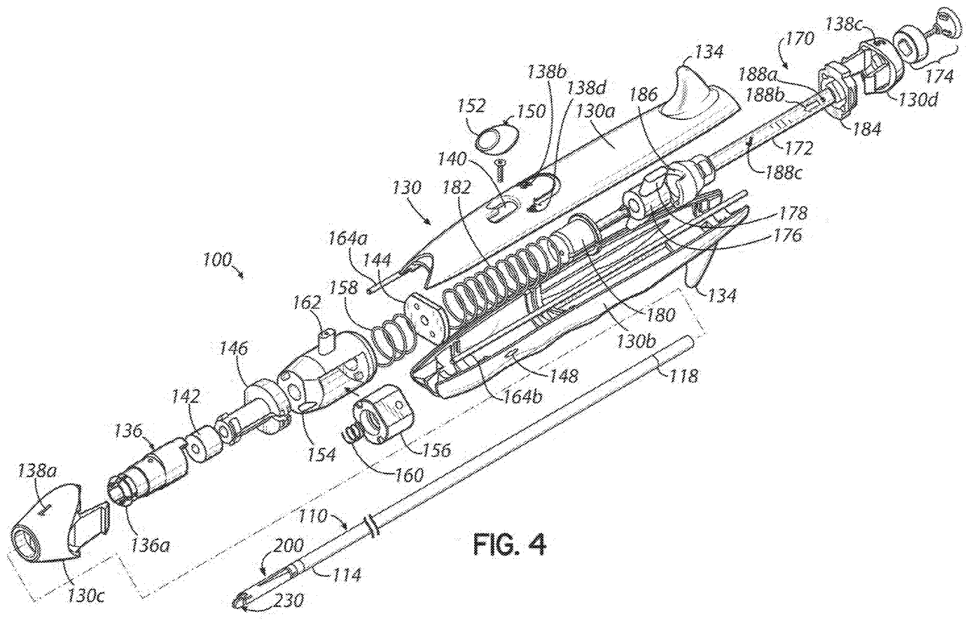

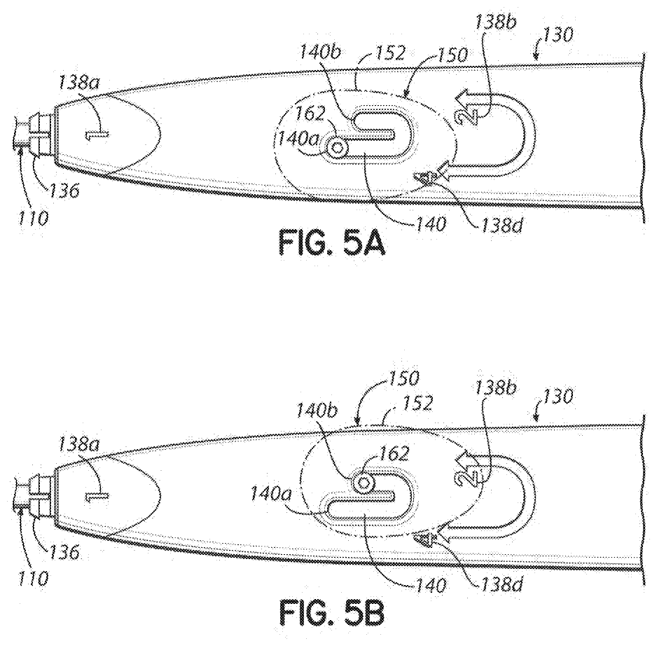

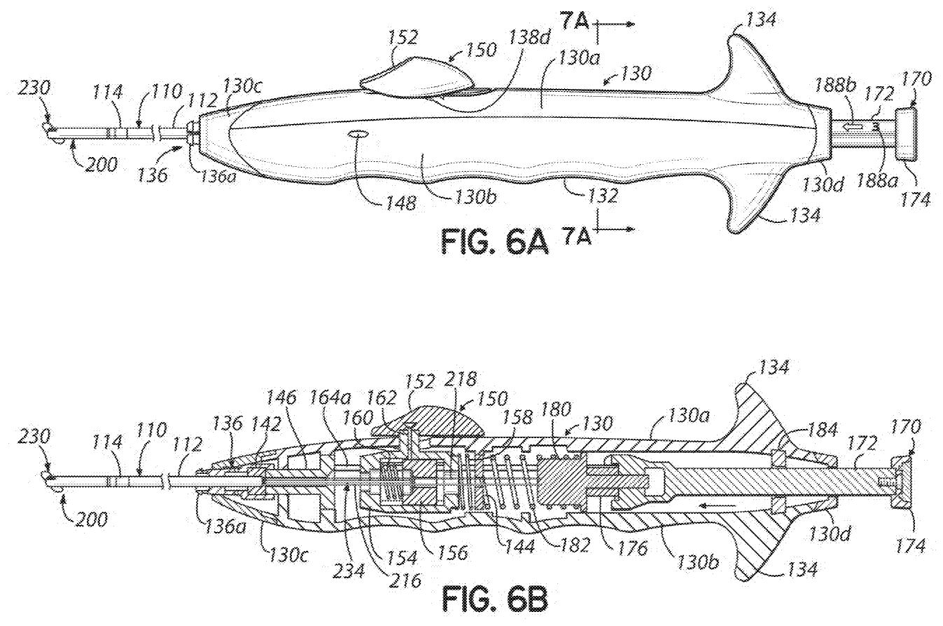

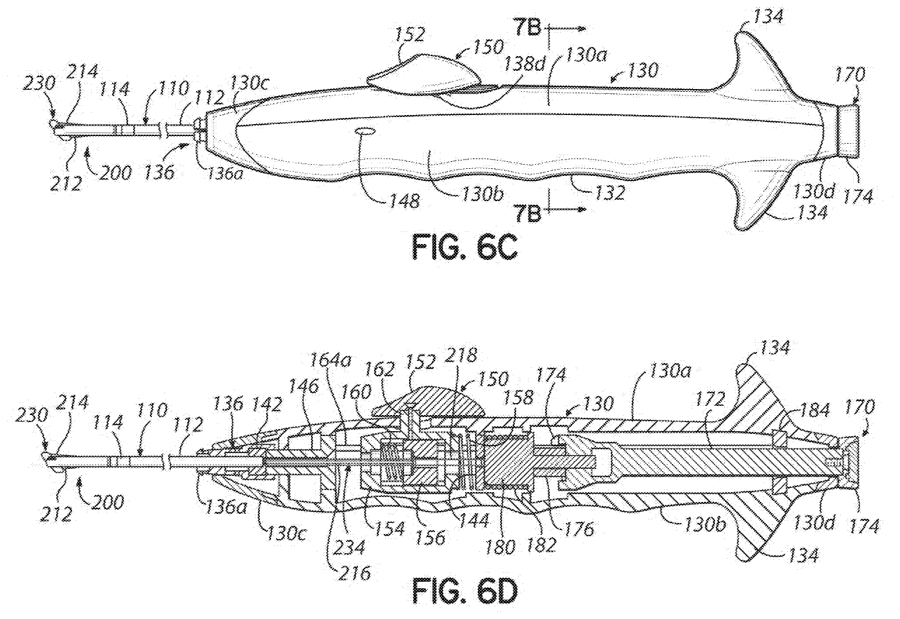

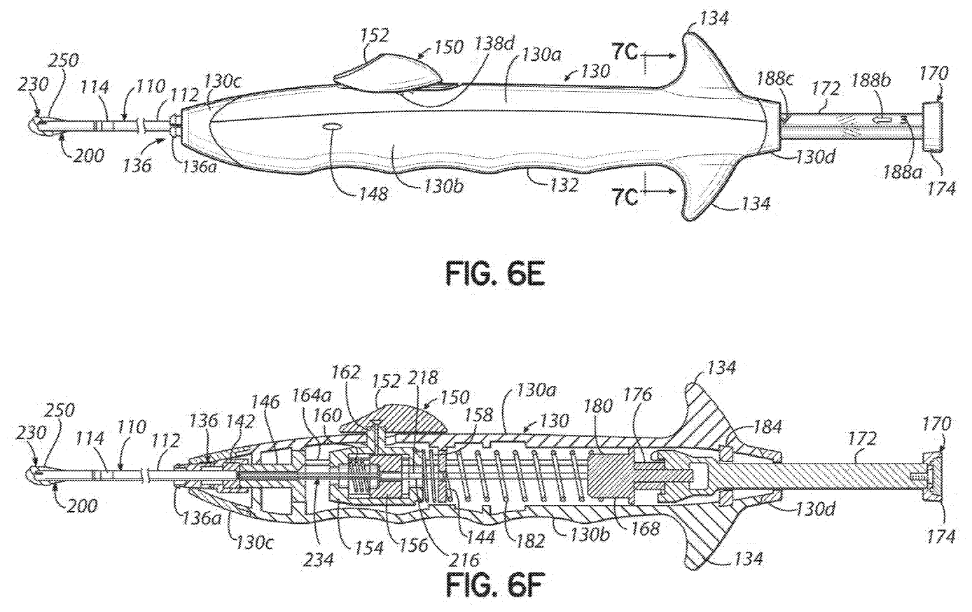

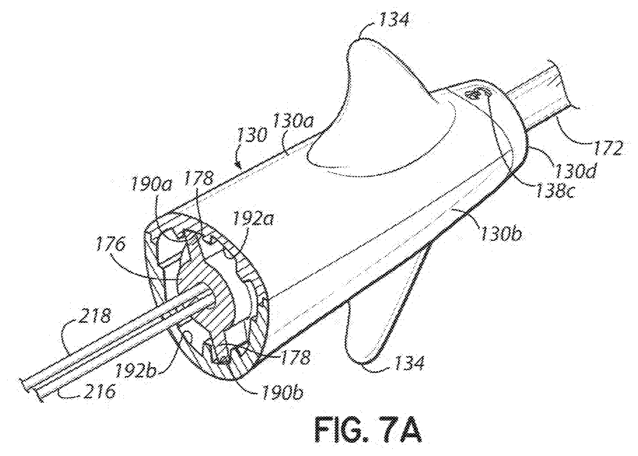

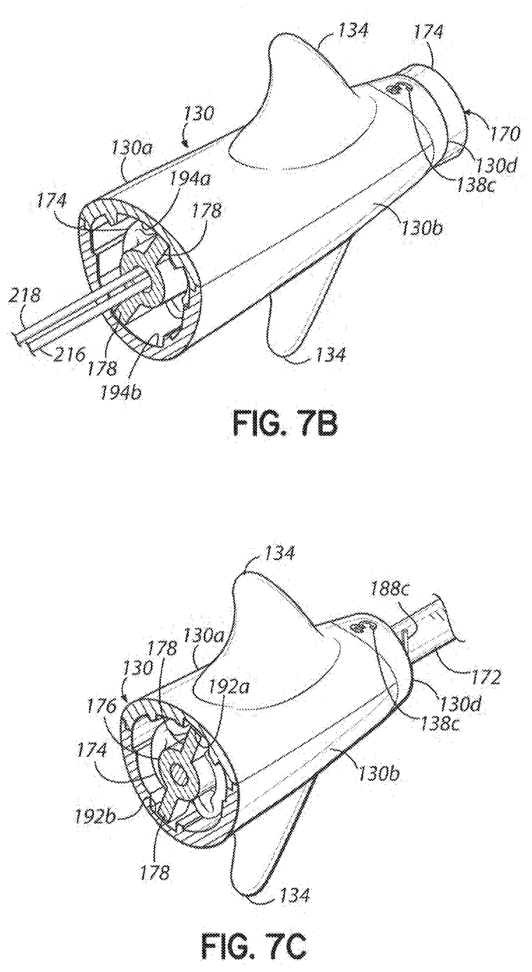

FIG. 4 is an exploded view of the suturing device 100 showing at least some of the components of the illustrative handle 130. FIGS. 5A and 5B are top views of a portion of the handle 130. FIGS. 6A, 6C, and 6E are side views of the illustrative suturing device 100 showing at least some of the external features of the handle 130. FIGS. 6B, 6D, and 6F are sectional side views of the suturing device 100 showing at least some of the components of the handle 130. FIGS. 7A, 7B, and 7C are sectional views of the handle 130.

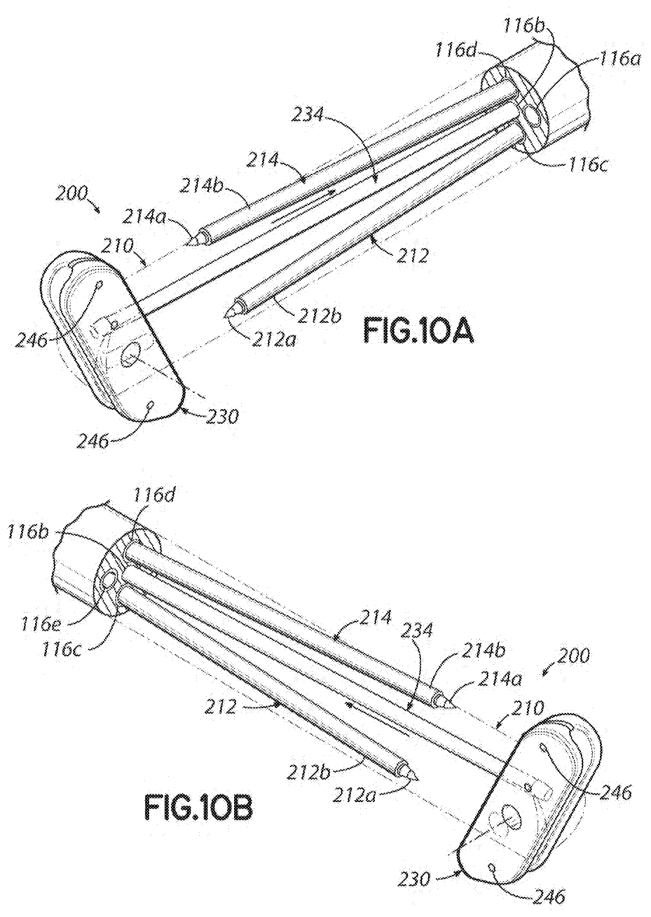

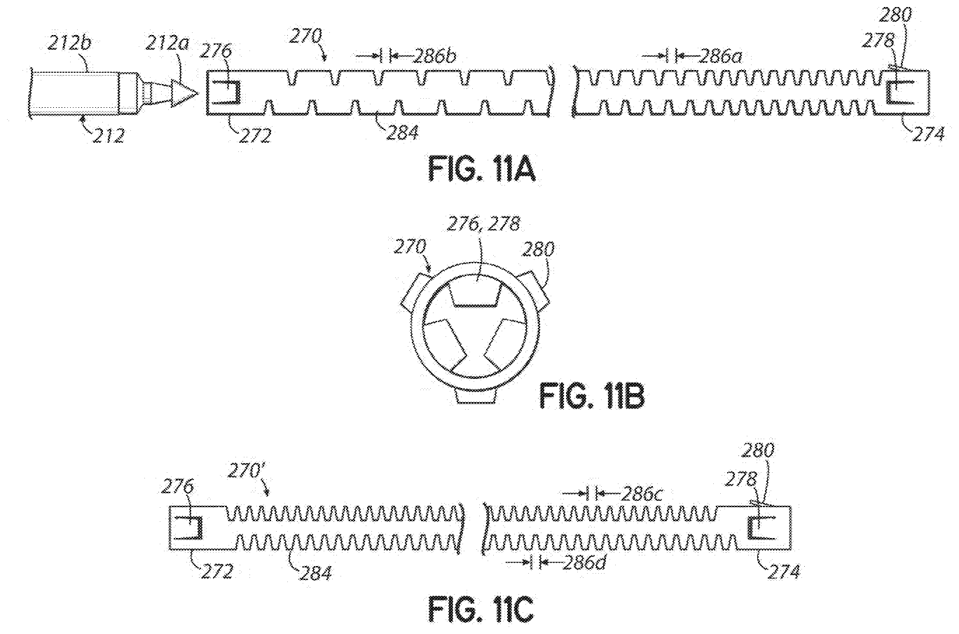

Referring to FIGS. 2, 4, and 6A, the handle 130 includes an upper shell 130a, a lower shell 130b, a nose cone 130c, and a rear cap 130d. The lower shell 130b includes a gripping surface with finger recesses 132 configured to assist a user griping the handle 130. The upper shell and lower shells, 130a, 130b include finger tabs 134 configured to assist a user operating the various features of the suturing device 100. A suturing mechanism actuator 150 configured to operate the suturing mechanism 200 is installed in the handle 130. A needle actuator 170 is located at the proximal end of the handle 130 and configured to deploy and retract the suturing needles 212, 214, see FIG. 10A and described herein below. The distal end of the handle 130 includes an insert 136 with locking tabs 136a for engaging and coupling the suturing device 100 with another device, such as an introducer sheath 20 for example, see FIG. 1. The handle 130 includes markings 138a, 138b, 138c, 138d which may include letters, numbers, and/or symbols, to assist and instruct a user operating the various features of the suturing device 100. The handle 130 includes a “J” shaped slot 140, a spring wall 144, a guide spacer 146, and a guidewire port 148. The shaft is connected to the distal end of the handle 130 and secured and sealed to the handle 130 with shaft seal 142, see FIG. 6B.

Referring to FIGS. 2, 4, 6A, and 6B, the suturing mechanism actuator 150 includes a selector knob 152, an actuator retainer 154, an actuator follower 156, and first and second suturing mechanism actuator springs 158, 160. Other forms of biasing members may be used in place of springs 158, 160. the actuator retainer 154 includes a suturing mechanism actuator shaft 162 that extends though the “J” shaped slot 140, see FIG. 3. The “J” shaped slot 140 guides the movement of the shaft 162, and thereby the movement of the actuator retainer 154, see FIG. 5A, and 5B. The first suturing mechanism actuator spring 158 biases the actuator retainer 154 in a distal direction as the spring 158 is compressed between the actuator retainer 154 and the spring wall 144. The distal bias of the actuator spring 158 holds the shaft 162 extending though the “J” shaped slot 140 in position at a first end 140a or a second end 140b of the “J” shaped slot 140 until a user actuates the selector knob 152. The actuator follower 156, which is located inside the actuator retainer 154, is biased in a proximal direction by the second suturing mechanism actuator spring 160. A pair of guide rods 164a, 164b guide and prevent the actuator follower 156 from rotating as it moves in the proximal and distal directions. While the suturing mechanism actuator 150 is shown and described as manually driven, the suturing mechanism actuator 150 may be motorized, mechanically leveraged, or assisted in other manners.

Referring to FIGS. 2, 4, 6E, and 6F, the needle actuator 170 includes a plunger 172, a plunger cap 174, a sprag 176, a needle follower 180, a spring 182, and a retainer 184. Other forms of biasing members may be used in place of spring 182. The plunger 172 includes a pair of angled guides 186 that engage tabs 178 on the sprag 176. The sprag tabs 178 alternately engage a first pair of sprag grooves 190a, 190b and a second pair of sprag grooves 192a, 192b connected by a third pair of sprag grooves 194a, 194b inside the handle 130, see FIGS. 7A, 7B, and 7C. The plunger 172 includes markings 188a, 188b, 188c, to assist and instruct a user operating the various features of the suturing device 100. The guide rods 164a, 164b guide and prevent the plunger 172 from rotating as it moves in the proximal and distal directions. While the needle actuator 170 is shown and described as manually driven, the needle actuator 170 may be motorized, mechanically leveraged, or assisted in other manners.

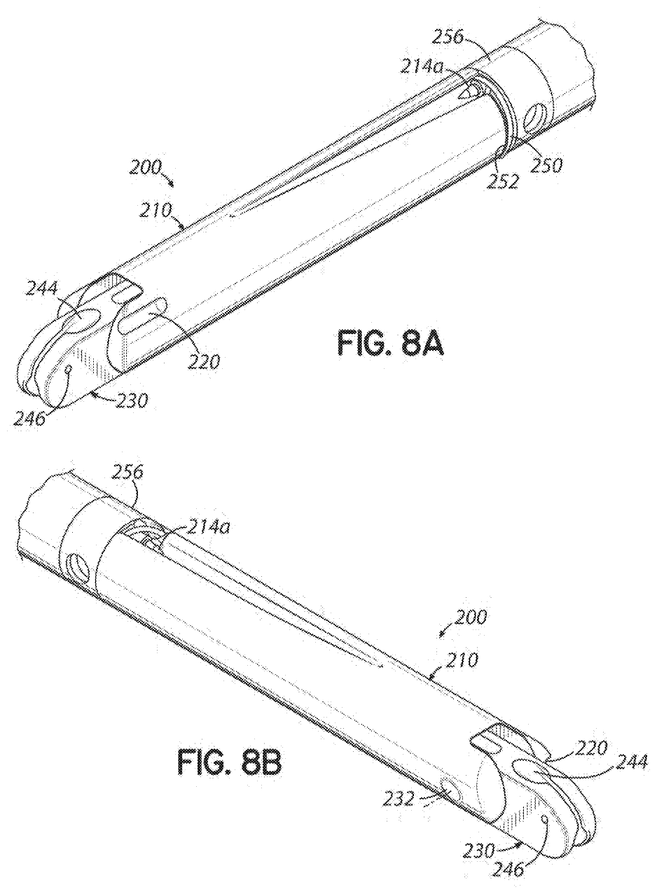

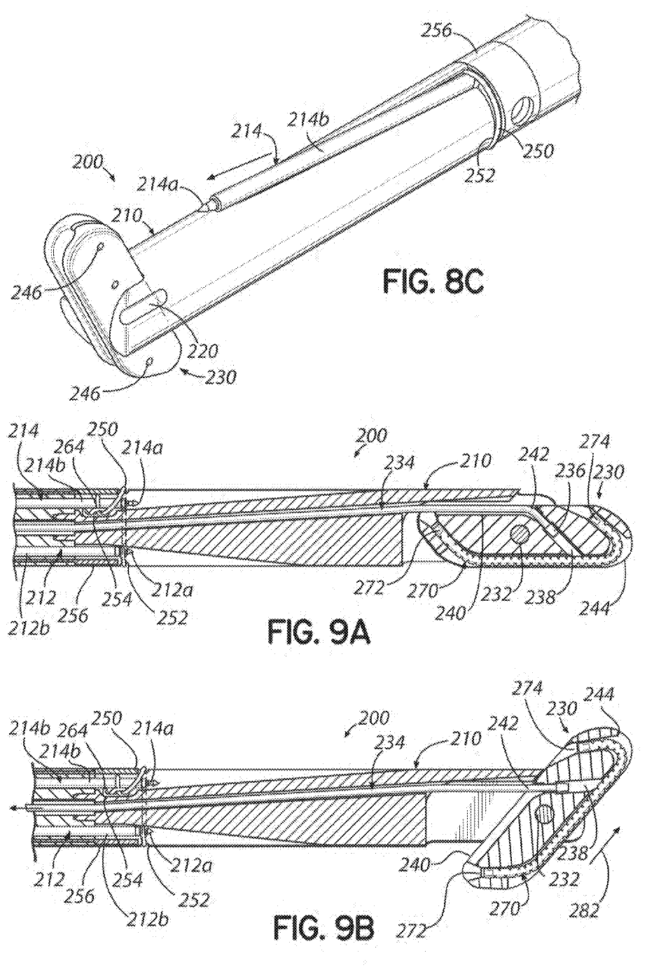

FIGS. 8A, 8B, and 8C are perspective views of the illustrative suturing mechanism 200, FIGS. 9A and 9B are sectional side views of the suturing mechanism 200, and FIGS. 10A and 10B are perspective views of the suturing mechanism 200 with portions of the suturing mechanism 200 hidden. The suturing mechanism 200 includes a needle guide 210 and a pivotal element 230. The needle guide 210 includes two needles 212, 214 which are either integral with or fixedly coupled to respective wires 216, 218, see FIGS. 7A and 7B. Needle 212 is a two-piece needle including a barbed needle tip 212a which is detachable from a needle body 212b by way of a friction fit or an adhesive, for example. Needle 214 has an integral (i.e., fixed) barbed needle tip 214a. The word “integral” here encompasses integral constructions as well as constructions in which the needle tip 214a is fixedly (i.e., not “removably”) coupled to the remainder of the integral needle 214. Wires 216, 218 may be, for example, solid wire-like members or hollow (e.g., hypotubes). Wires 216, 218 are coupled to the needle follower 180 of the needle actuator 170, see FIG. 7B. The needle guide 210 also includes a guidewire lumen 220 which is coupled to the guidewire lumen 116a of the shaft 110, see FIG. 10A, which is coupled to the guidewire port 148 in the handle 130, see FIG. 2. The shaft 110 may be a multi-lumen flexible or rigid member in which a central lumen 116b carries the actuator wire 234, two of the lumens 116c, 116d respectively carry the needles 212, 214, and a fifth lumen 116e may be used for a guidewire, for flushing, and/or as a blood port. When used as a blood port, lumen 116e may provide an indication to the user that the distal end of the suturing device 100 and/or the distal end of the introducer sheath 20 has entered the blood vessel 60. In some embodiments, the needle guide 210 may be an integral part of the shaft 110.

The pivotal element 230 is secured to the distal end of the needle guide 210 by a pivot 232. Pivot 232 allows the pivotal element 230 to pivot between an insertion and removal orientation, shown in FIGS. 8A, 8B, and 9A, and a deployed orientation, shown in FIGS. 8C and 9B. The insertion and removal orientation generally aligns the pivotal element 230 with the needle guide 210 and the shaft 110. The insertion and removal orientation allows the pivotal element 230 to be inserted into and removed from a blood vessel, for example. The deployed orientation orients the pivotal element 230 at an angle to the lengthwise axes of the needle guide 210 and the shaft 110. In some embodiments, the deployed orientation orients the pivotal element 230 at an angle that is generally or nearly perpendicular to the lengthwise axes of the needle guide 210 and the shaft 110. In other embodiments, the angle of the deployed pivotal element 230 to the lengthwise axes of the needle guide 210 and the shaft 110 may be between 30 and 90 degrees. The deployed orientation of the pivotal element 230 facilitates a suturing procedure performed from within a blood vessel, for example.

Referring to FIGS. 6B, 9A, and 9B, in this illustrative embodiment, the suturing mechanism actuator 150 is coupled to the pivotal element 230 by an actuating wire 234. The actuator wire 234 is coupled to the actuator follower 156 of the suturing mechanism actuator 150. The actuator wire 234 includes a retainer 236 that engages a hole 238 in the pivotal element 230 and secures the actuator wire 234 to the pivotal element 230. The pivotal element 230 includes an actuation wire groove 240 configured to accommodate the actuation wire 234 when the pivotal element 230 is in the insertion and removal orientation as shown in FIG. 9A. The distal end of the actuation wire 234 includes a formed angle 242. The actuation wire 234 may be heat-set to create the formed angle 242, for example. The formed angle 242 acts as a biasing member to hold the pivotal element 230 in lengthwise alignment with the lengthwise axis of the shaft 110 for suturing mechanism 200 insertion and removal. The pivotal element 230 includes a passage 244 with an flexible coupling member 270 contained therein. The pivotal element 230 also includes through holes 246 that are used to secure the flexible coupling member 270. During assembly of the flexible coupling member 270 to the pivotal element 230, an adhesive, such as an epoxy for example, is injected into the holes 246 thereby securing the flexible coupling member 270 to the pivotal element 230. The flexible coupling member 270 may be secured to the pivotal element 230 by other means such as a friction fit between the flexible coupling member 270 and the passage 244.

When a user moves the selector knob 152, the “J” shaped slot 140 limits and guides the movement of the suturing mechanism actuator shaft 162, and thereby the movement of the actuator retainer 154, see FIGS. 5A, 5B and 6B. The actuator retainer 154 can be moved in both proximal and distal directions relative to the handle 130 and rotated around a central axis of the handle 130 by moving the selector knob 152. Generally, moving the selector knob 152 in a proximal direction moves the actuator follower 156 in a proximal direction which tensions the actuator wire 234 thereby moving the pivotal element 230 into the deployed orientation, shown in FIG. 6B. Generally, moving the selector knob 152 in a distal direction moves the actuator follower 156 in a distal direction which reduces tension on and/or compresses the actuator wire 234 thereby moving the pivotal element 230 into the insertion and removal orientation, shown in FIG. 6A. When the suturing mechanism actuator 150 is in a first position, with the suturing mechanism actuator shaft 162 in the first end 140a of the “J” shaped slot 140 as shown in FIG. 5A, the pivotal element 230 of the suturing mechanism 200 is in the insertion and removal orientation as shown in FIG. 9A. When the suturing mechanism actuator 150 is in a second position, with the suturing mechanism actuator shaft 162 in the second end 140b of the “J” shaped slot 140 as shown in FIG. 5B, the pivotal element 230 is in the deployed orientation as shown in FIG. 9B.

Referring to FIGS. 8A, 8B, 8C, 9A, and 9B, in this illustrative embodiment, the needle guide 210 includes a tensile member 250 in tensile member groove 252 that wraps around the needle guide 210 from the first needle 212 side to the second needle 214 side. One end of tensile member 250 is coupled to needle tip 212a. The needle guide 210 also includes a knot recess 254 containing a suture knot 264. The knot recess 254 is a cavity in the needle guide large enough to accommodate the suture knot 264. A ring 256 wraps around the proximal end of the needle guide 210, coupling the needle guide 210 to the shaft 110. The ring 256 may partially cover the knot recess 254. The “coils” of the suture knot 264 are wrapped around the needle 214 and the needle 214 holds the suture knot 264 in the knot recess 254 until the suture knot 264 needs to be deployed. The suture knot 264 may be any desired form of knot suitable for tying off a suture, such as a sliding knot that will automatically slide along the tensile member 250 and tighten as one end of the tensile member 250 is pulled. One form of knot suitable for use in this situation is a “Tennessee Slider” knot.

As described herein, the length of the tensile member 250 is the measured distance from the last loop or coil of the suture knot 264 to the end of the tensile member 250 that is coupled to needle tip 212a. The length of the tensile member 250 determines the deployment distance for the suture knot 264 which is the distance that the suturing mechanism 200 must be retracted from the percutaneous exit site of the patient before the suture knot 264 is deployed from the suturing mechanism 200. A short tensile member length means that the suture knot 264 is released very close to the percutaneous exit site of the patient. A long tensile member length means that the suture knot 264 is released further from the percutaneous exit site of the patient. The tensile member length may be from 1.0″ to 10.0″.

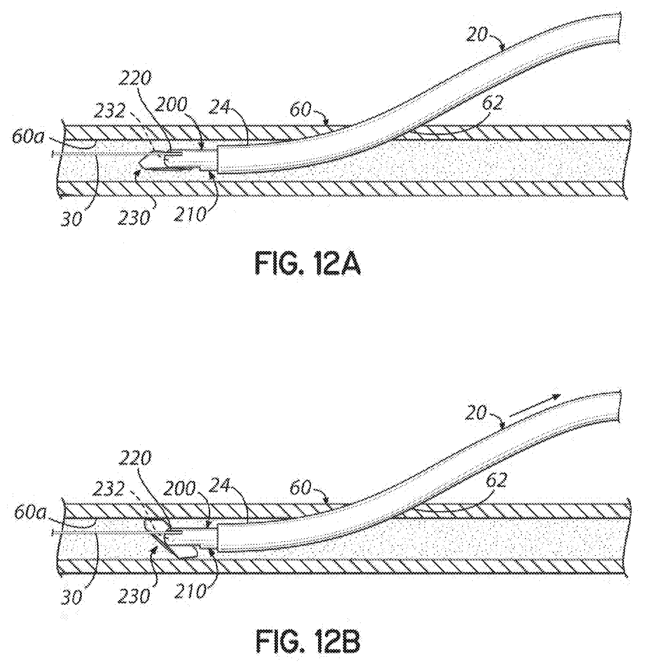

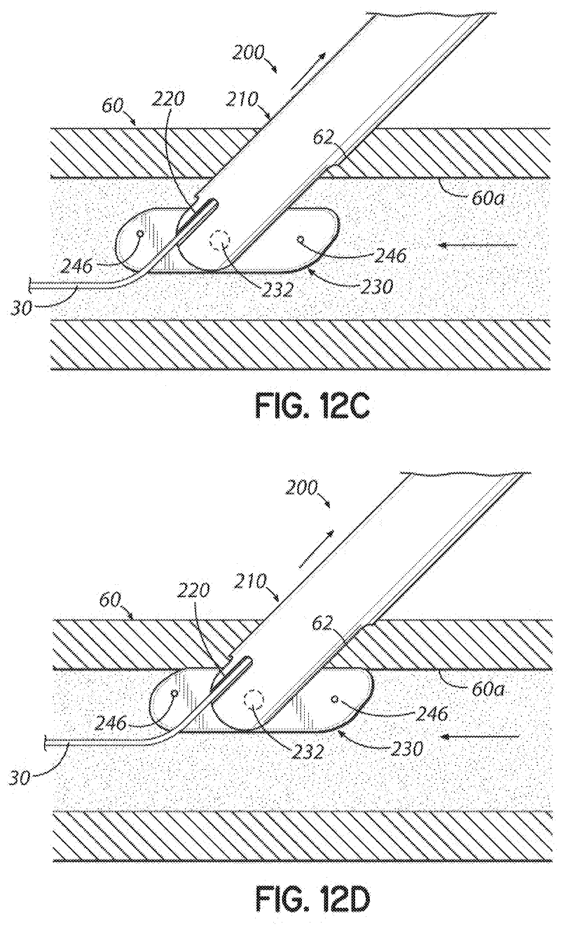

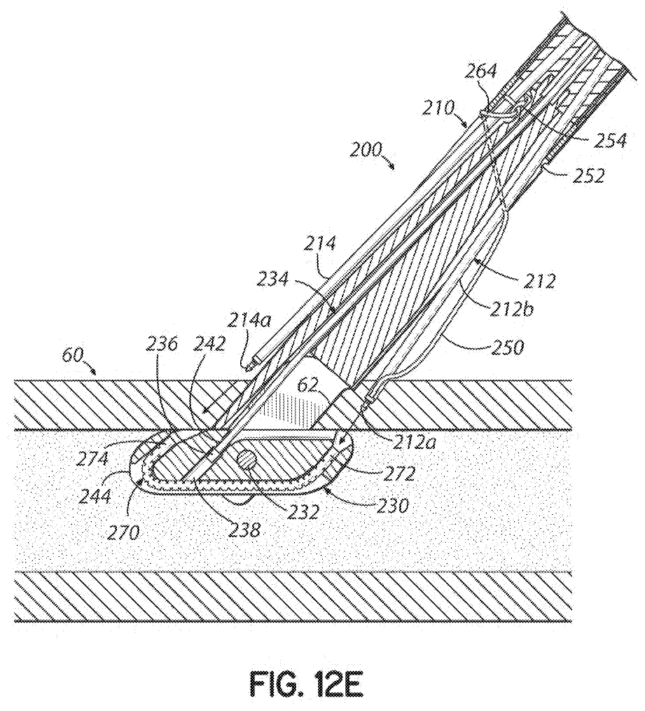

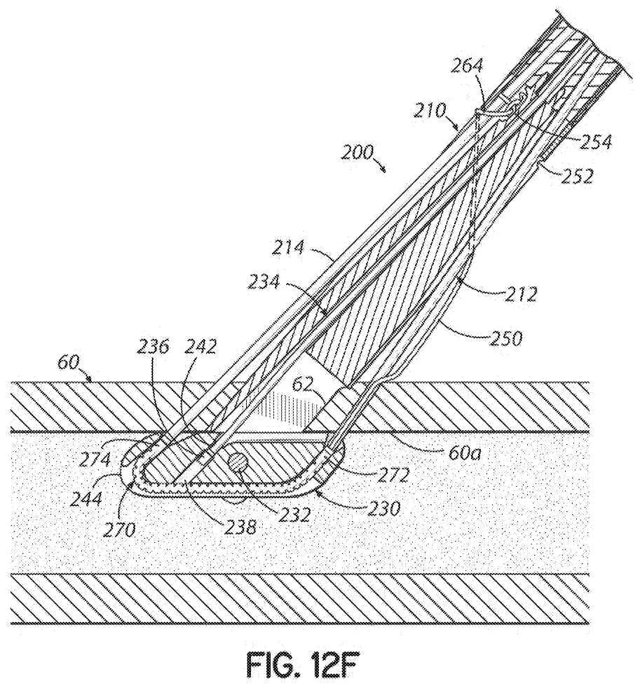

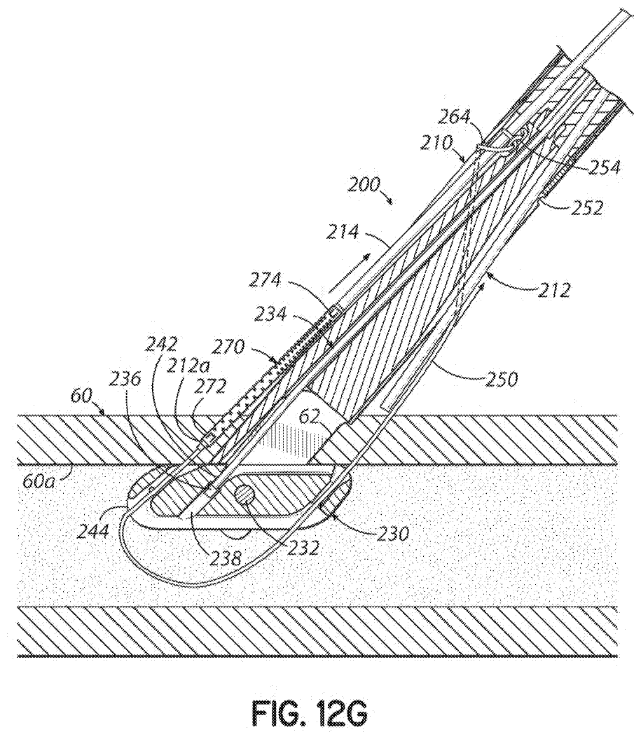

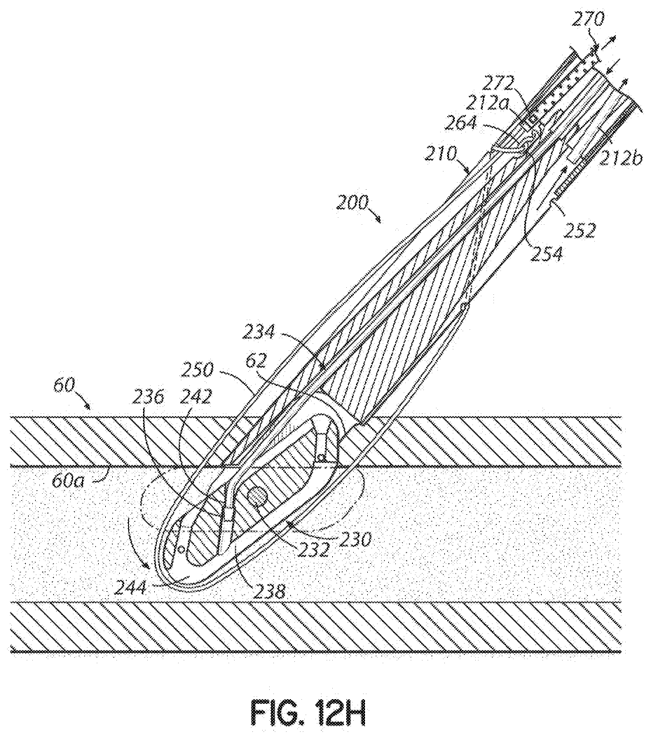

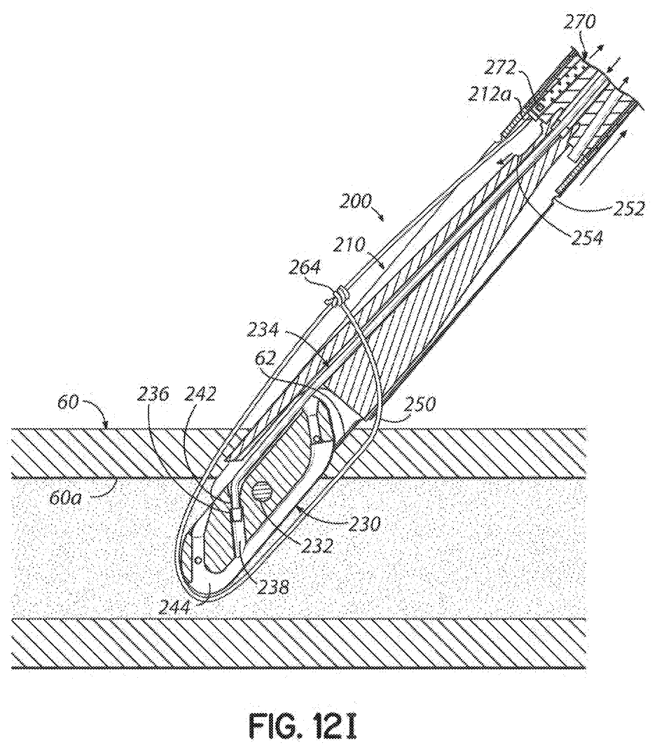

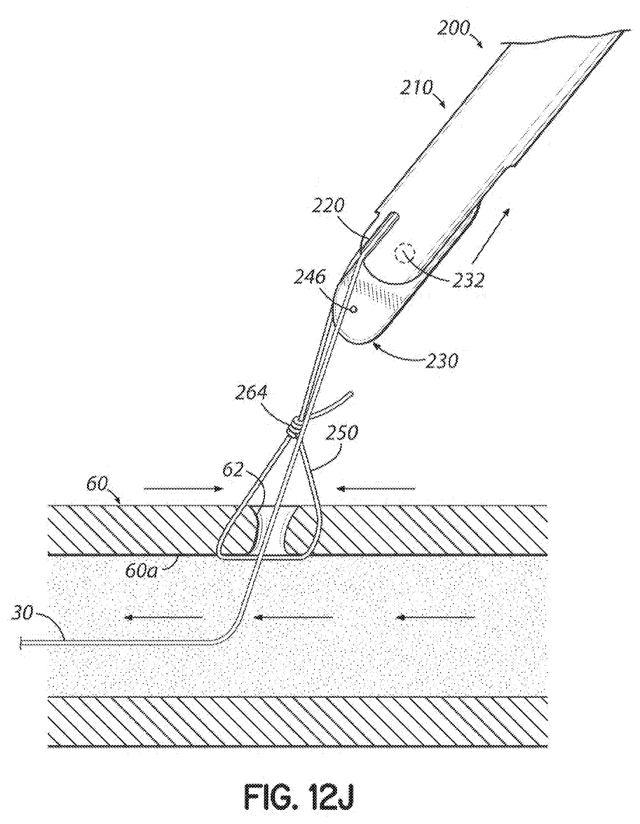

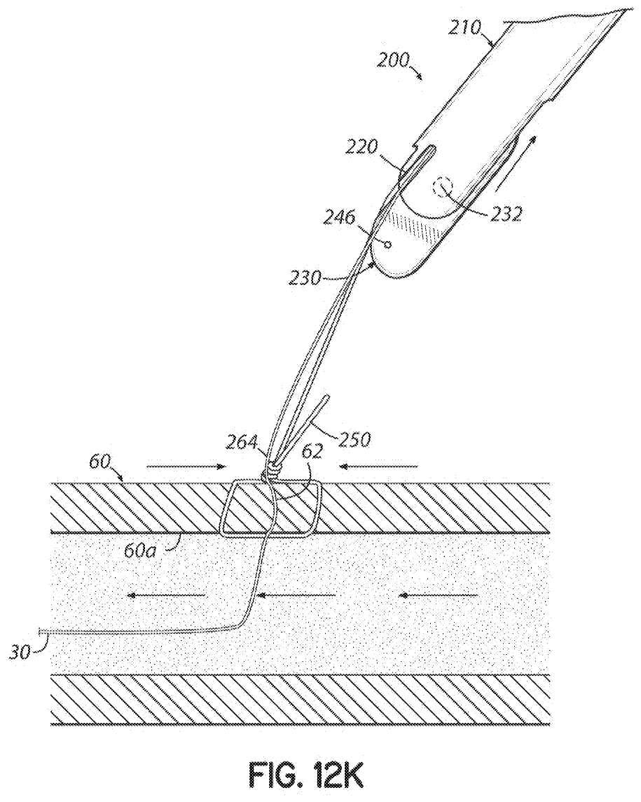

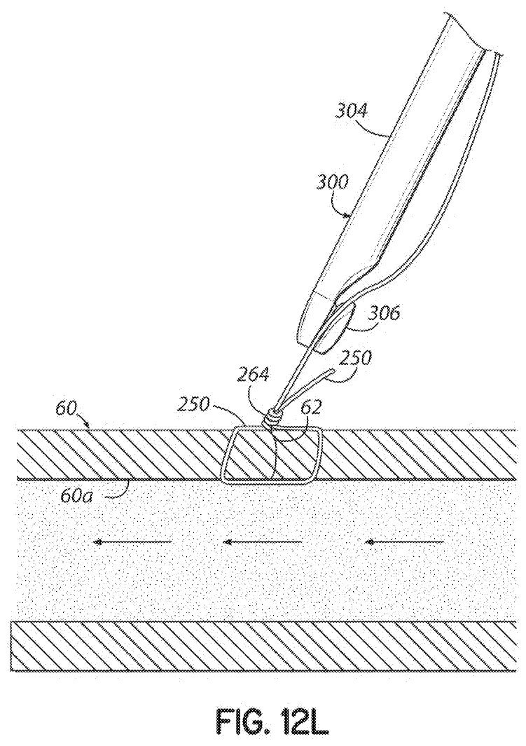

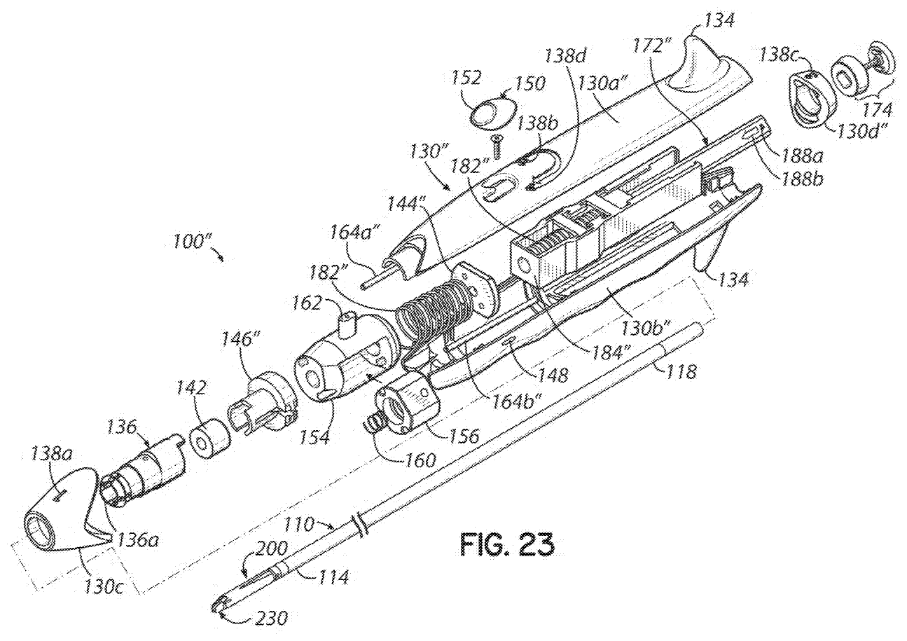

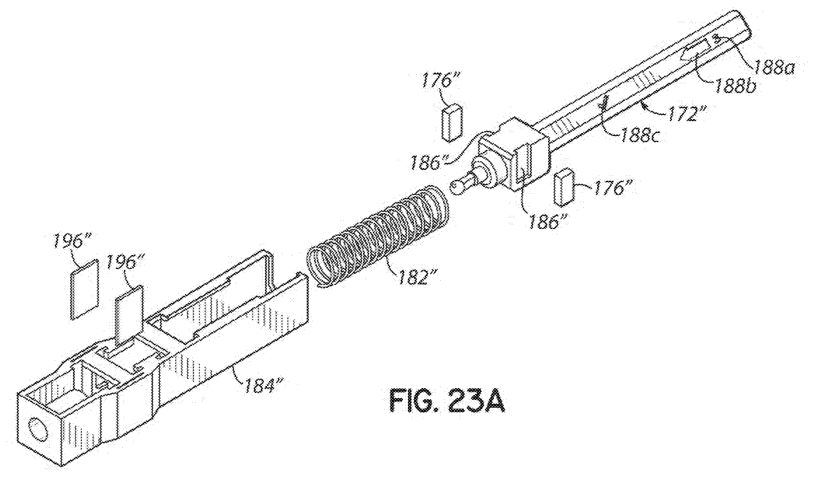

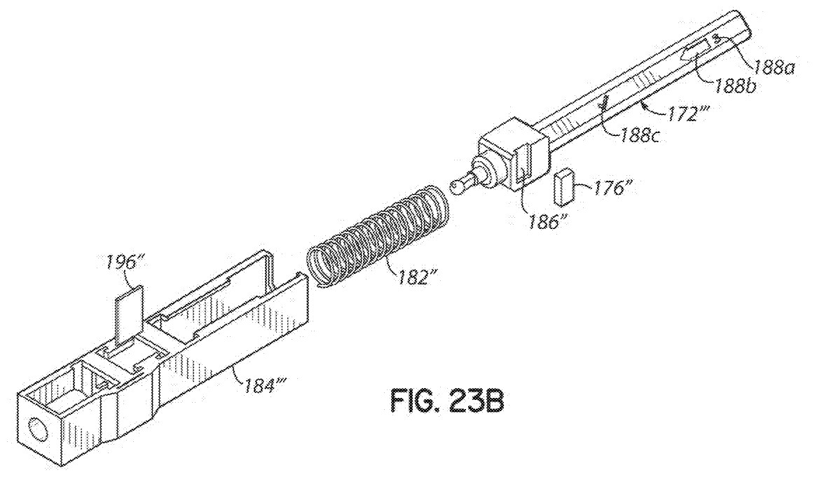

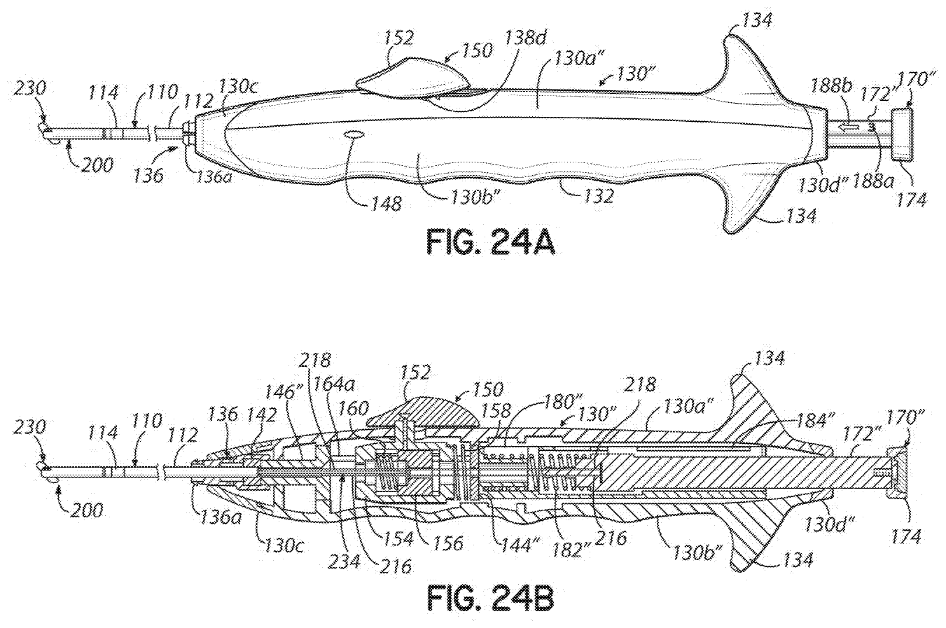

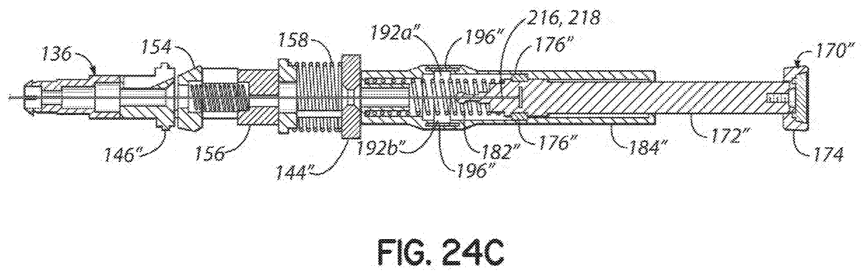

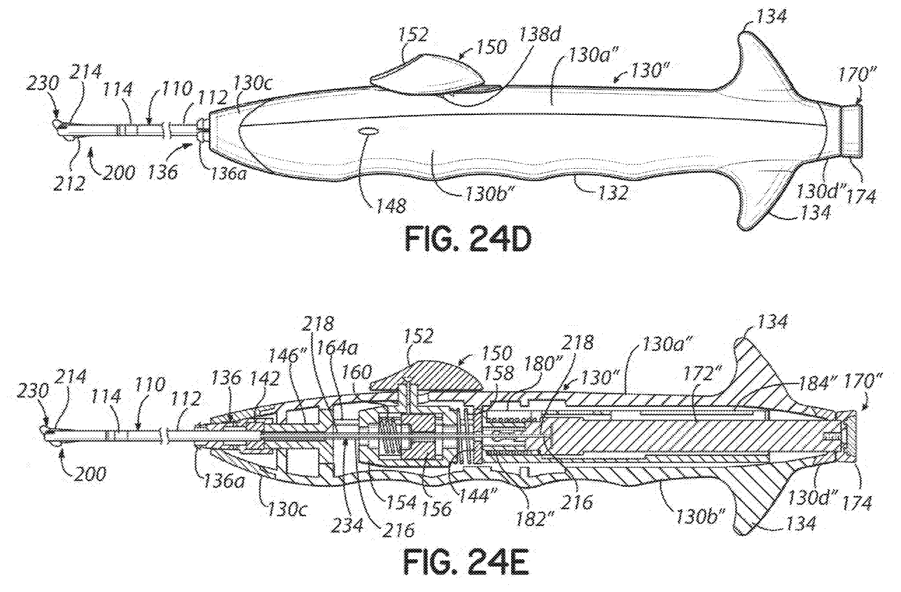

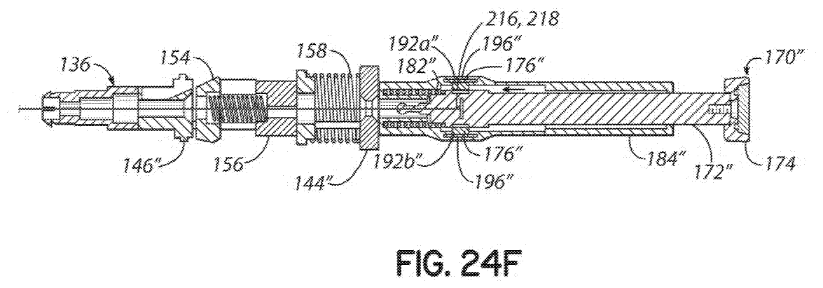

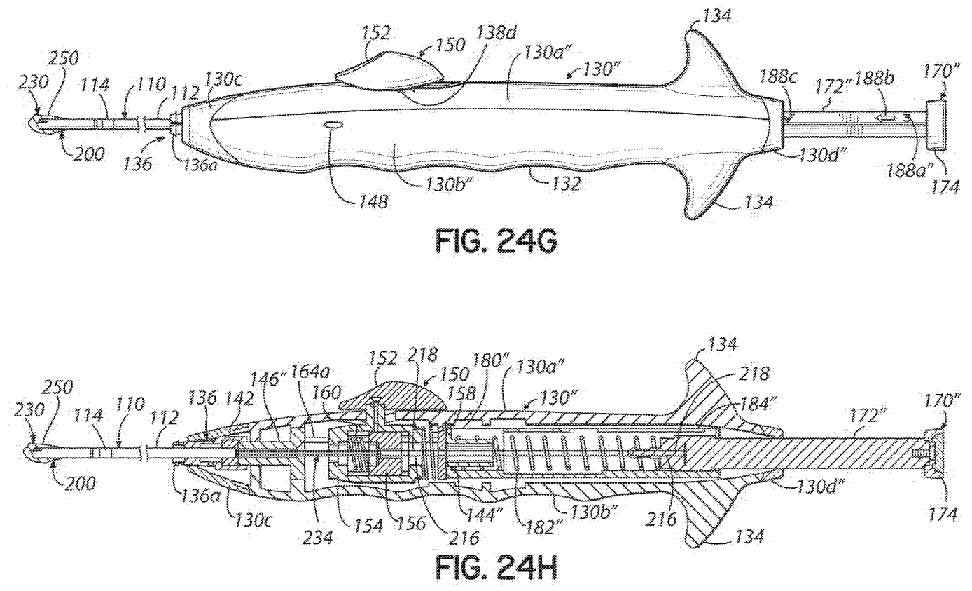

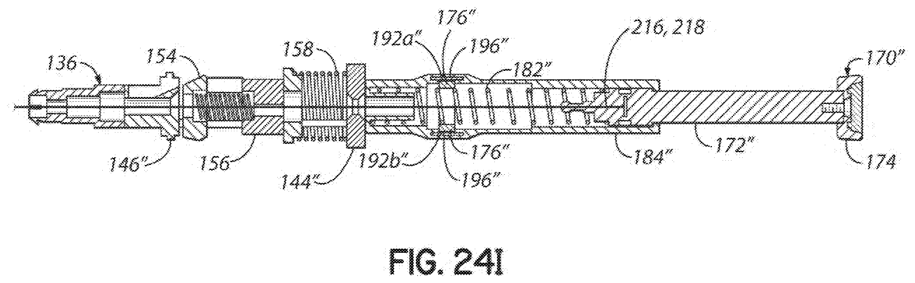

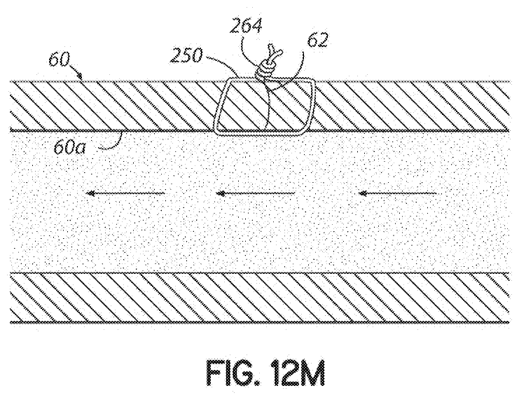

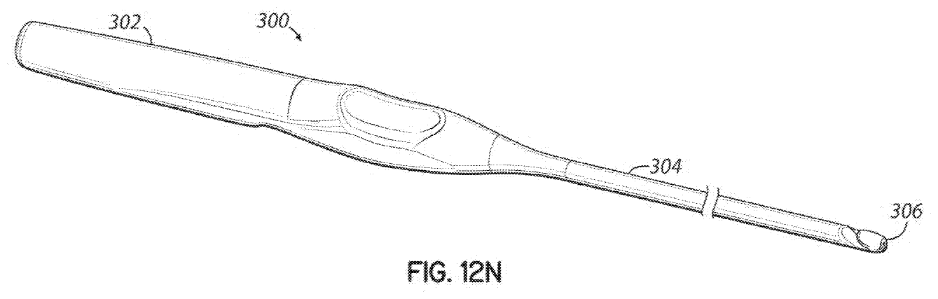

FIG. 11A is a side view of an illustrative coupling member 270 and FIG. 11B is an end view of the coupling member 270. The coupling member 270 has first and second end portions 272, 274. The first and second end portions 272, 274 include internal tabs 276, 278 configured to grip needle tips 212a, 214a respectively thereby coupling needle tips 212a, 214a to the coupling member 270, see FIGS. 12E and 12F. The coupling member 270 is configured to pull the tensile member 250 through tissue during a suturing procedure for closing an opening in a blood vessel wall described herein. The coupling member 270 may also have external tabs 280 configured so that coupling member 270 can only move in one direction, indicated by arrow 282, in the passage 244 of the pivotal element 230, see FIG. 9B. The tabs 280 may be fixed or movable to allow for the one-way movement of the coupling member 270 in the passage 244 and prevent the coupling member 270 from backing out of the passage 244.

In this illustrative embodiment, the coupling member 270 includes a plurality of opposing notches 284 configured to allow the coupling member 270 to bend or deform in a single plane. Limiting the deformation of the coupling member 270 to a single plane may improve the control of the coupling member 270. Locating the notches 284 on opposing sides of a central axis of the coupling member 270, as opposed to a spiral wound member for example, allows the coupling member 270 to deform without elongating the coupling member 270. The notches 284 are spaced along the length of the coupling member 270, and the spacing of the notches at least partially defines a bend radius of the coupling member 270. Varying the spacing of the notches 284 along the length of the coupling member 270 will create a plurality of bend radii. The notches 284 have widths 286a, 286b, and the widths 286a, 286b of the notches 284 at least partially define a bend radius of the coupling member 270. The notches 284 may have a plurality of widths 286a, 286b creating a plurality of bend radii. Alternatively, the notches 284 along the length of the coupling member 270 can patterned in a helical fashion with a plurality of pitch distances.

FIG. 11C is a side view of an alternative illustrative coupling member 270′. Generally, the coupling member 270′ is similar in construction and operation to the coupling member 270 described above and the coupling member 270′ may be substituted for other coupling members, or any feature of the coupling member 270′ may be used, in various other exemplary embodiments according to the present disclosure. Like reference numbers refer to like components. For brevity, the following description minimizes redundant description and focuses on the differences between the coupling member 270 and the coupling member 270′. In this illustrative embodiment, the coupling member 270′ includes a plurality of opposing notches 284′ configured to allow the coupling member 270′ to bend or deform in a single plane. The notches 284′ are equally spaced along the length of the coupling member 270′, and the spacing of the notches at least partially defines a bend radius of the coupling member 270′. The notches 284′ have uniform widths 286′, and the widths 286′ of the notches 284′ at least partially define a bend radius of the coupling member 270′.

FIGS. 12A through 12M progressively illustrate the use of the suturing device 100 and, particularly, the distal end and the suturing mechanism 200 within a blood vessel 60, such as the common carotid artery, for closing a puncture or opening 62 in a vessel wall 60a of the blood vessel 60. The opening 62 may be a vessel access site for a medical procedure. FIG. 12N is a perspective view of an illustrative knot pusher 300.

In preparation for a procedure, a guidewire 30, such as a 0.018″ OD guidewire, for example, is inserted in through the guidewire port 148 in the handle 130 and out through the guidewire lumen 220 in the suturing mechanism 200, see FIGS. 2 and 8A. Alternatively, the guidewire 30 may be inserted in through the guidewire lumen 220 in the suturing mechanism 200 and out through the guidewire port 148 in the handle 130. The guidewire 30 and the suturing mechanism 200, with the pivotal element 230 in the insertion and removal orientation, is inserted into the proximal end 22 of the introducer sheath 20, see FIG. 1. The introducer sheath 20 is guided through the opening 62 in the blood vessel 60 using the guidewire 30. The suturing mechanism 200 may be directed over the guidewire 30 by inserting the guidewire 30 into the guidewire lumen 220 and out through the guidewire port 148 in the handle 130, see FIGS. 2 and 8A.

The suturing device 100 is advanced until the mark 118 on the shaft 110 is aligned with the proximal end 22 of the introducer sheath 20, see FIGS. 1 and 2. When the mark 118 on the shaft 110 is aligned with the proximal end 22 of the introducer sheath 20, the suturing mechanism 200 is entirely within the introducer sheath 20. The suturing mechanism 200 and the shaft 110 are held stationary relative to the vessel 60 and the introducer sheath 20 is pulled proximally over suturing mechanism 200 and shaft 110 until the locking tabs 136a engage the proximal end 22 of the introducer sheath 20, see FIGS. 1 and 2. This corresponds to marking 138a, which in this illustrative example is “1” indicating step 1 to a user. The locking tabs 136a secure the suturing device 100 to the introducer sheath 20 with the suturing mechanism 200 extending past the distal end 24 of the introducer sheath 20 and into the blood vessel 60, see FIG. 12A. Coupling the locking tabs 136a to the distal end 22 of the introducer sheath 20 ensures the suturing mechanism 200 extends past the distal end 24 of the introducer sheath 20. The introducer sheath 20 is withdrawn from the opening 62 in the blood vessel 60 while the suturing mechanism 200 remains inside the blood vessel 60 thereby allowing an internal exchange of the suturing device 100 and the introducer sheath 20. This will inhibit (eliminate, or at least substantially reduce) blood loss normally experienced during a typical device exchange as a result of the high blood pressures found in the common carotid artery, for example. The introducer sheath 20 remains in surrounding relation to the shaft 110 for the remainder of the procedure.

Alternatively, the distal end 24 of the introducer sheath 20 may be inside the blood vessel 60 as part of a procedure, for example. The distal end of the needle guide 210, with the pivotal element 230 aligned with its lengthwise extent parallel to and in alignment with the lengthwise axis of the needle guide 210 and the shaft 110, is inserted into the proximal end 22 of the introducer sheath 20. Depth markers or indicators 118 on the shaft 110 give visual feedback to the user as to how far the suturing device 100 is inserted into the introducer sheath 20. The suturing device 100 is advanced until the locking tabs 136a engage the proximal end 22 of the introducer sheath 20, see FIG. 1. As described above, the locking tabs 136a secure the suturing device 100 to the introducer sheath 20 with the suturing mechanism 200 extending past the distal end of the introducer sheath 20.

The arrow and “2” of marking 138b, indicate step 2 to a user. With the distal end of the needle guide 210 inside the blood vessel 60, the selector knob 152 is moved in a first direction indicated by the arrow and “2” of marking 138b, see FIG. 5B, moving the shaft 162 from a position at the first end 140a to the second end 140b of the “J” shaped slot 140, see FIGS. 5A and 5B, which causes the pivotal element 230 to pivot or rotate from the insertion and removal orientation to the deployed orientation, see FIG. 12B. The ‘J’ slot 140 helps guide the movement of the selector knob 152. Moving the selector knob 152 moves the actuator retainer 154 and actuator follower 156 against the bias provided by springs 158, 160, see FIG. 6B. Moving selector knob 152 also rotates the actuator retainer 154 in a first direction. The bias of spring 158 holds the actuator retainer 154 in position in the ‘J’ slot 140. The pivotal element 230 may be rotated into the deployed orientation before the introducer sheath 20 is withdrawn from the opening 62 in the blood vessel 60, see FIG. 12B, or after the introducer sheath 20 is withdrawn from the opening 62 in the blood vessel 60, see FIG. 12C. The pivotal element 230 is then pulled proximally against the interior of the vessel wall 60a by slightly pulling proximally on the suturing device 100 and therefore the needle guide 210 as shown in FIG. 12D. The deployed orientation of the pivotal element 230 ensures the pivotal element 230 remains inside the blood vessel 60.

The next step is indicated to a user by the “3” of marking 138c, See FIG. 2. While the pivotal element 230 is in the deployed orientation, suture needles 212, 214 are deployed or moved in a distal direction, see FIG. 12E, by pushing the plunger cap 174, and thereby the plunger 172, sprag 176, and needle follower 180 of the needle actuator 170, in a distal direction as indicated by the arrow 188b, see FIGS. 6A and 6C and against the bias provided by spring 182, see FIGS. 6B and 6D. The needle follower 180 moves suture needles 212, 214 in a distal direction toward the coupling member 270 attached to the pivotal element 230. Needle tip 212a pulls tensile member 250 distally and out of the tensile member groove 252 and then engages and connects with the first end 272 of coupling member 270, see FIG. 12F. Needle tip 214a engages and connects with the second end 274 of coupling member 270.

As the needle actuator 170 moves in the distal direction, the angled guides 186 of the plunger 172 engage the tabs 178 of the sprag 176, see FIG. 4. The plunger 172 moves the sprag 176 in a distal direction with the sprag tabs 178 in the first pair of sprag grooves 190a, 190b inside the handle 130 guiding the sprag 176, see FIG. 7B. When the sprag tabs 178 reach the distal end of the first pair of sprag grooves 190a, 190b, the angled guides 186 cause the sprag 176 to rotate in sprag grooves 194a, 194b, see FIG. 7A, and the sprag tabs 178 to engage the second pair of sprag grooves 192a, 192b, inside the handle 130, see FIG. 7C.

Releasing the plunger cap 174, and thereby the plunger 172, allows the bias of spring 182 to move the needle follower 180 and sprag 176 in a proximal direction, see FIGS. 6E and 6F. This action pulls the suture needles 212, 214 in a proximal direction, see FIG. 12G. Suture needle 214 pulls coupling member 270 through the passage 244 of the pivotal element 230 and through the vessel wall 60a. Pulling or pushing the flexible coupling member 270 overcomes the bonding force of the adhesive or the friction fit securing the flexible coupling member 270 to the pivotal element 230. As coupling member 270 is pulled by suture needle 214, needle tip 212a is detached from needle body 212b of suture needle 212. Needle tip 212a is pulled by coupling member 270 and pulls tensile member 250 through the vessel wall 60a. Suture needle 214 pulls coupling member 270, needle tip 212a, and tensile member 250 through the suture knot 264, see FIGS. 12H and 121. As the sprag 176 moves in the proximal direction, the second pair of sprag grooves 192a, 192b inside the handle 130 guide the sprag tabs 178. The second pair of sprag grooves 192a, 192b allow the plunger 172 to extend further from the proximal end of the handle 130, see FIG. 6E. This exposes the checkmark symbol “V” marking 188c, which indicates to the user that the needles 212, 214 are retracted.

Step 4 is indicated to the user by the arrow and “4” of marking 138d. Next, the pivotal element 230 is moved distally away from the interior of the vessel wall 60a and then rotated from the deployed orientation to the insertion and removal orientation, see FIG. 12H. Rotating the pivotal element 230 is accomplished by moving the selector knob 152 in a second direction indicated by the arrow and “4” of marking 138d, see FIG. 5A, moving the shaft 162 from a position at the second end 140b to the first end 140a of the “J” shaped slot 140, see FIGS. 5A and 5B. Moving the selector knob 152 moves the actuator retainer 154 and actuator follower 156 against the bias provided by springs 158, 160. Moving selector knob 152 also rotates the actuator retainer 154 in a second direction. The bias of spring 158 holds the actuator retainer 154 in position in the ‘J’ slot 140. With the pivotal element 230 in the insertion and removal orientation the suturing device 100 is pulled in the proximal direction thereby removing the suturing mechanism 200 from the blood vessel 60 and releasing the suture knot 264 from the knot recess 254, see FIG. 12I. As the suturing mechanism 200 is pulled in the proximal direction the suture knot 264 slides along the tensile member 250 towards the opening 62 in the blood vessel 60. As the suturing mechanism 200 is pulled proximally, the suture knot 264 is tightened against the outside of the vessel 60, see FIGS. 12J and 12K, to approximate or close the opening 62. The guidewire 30 remains in the opening 62 while the suture knot 264 is tightened against the outside of the vessel 60 and the guidewire 30 is not removed until the opening 62 is closed. During some procedures it may be necessary to push the suture knot 264 toward the opening 62. Referring to FIGS. 12L and 12N, a knot pusher 300, including a handle 302, an elongated shaft 304, and loop or ring 306, may be used to push the suture knot 264 toward the opening 62. Finally, the ends of tensile member 250 outside of the suture knot 264 are cut off, leaving the opening 62 securely closed and sealed as shown in FIG. 12M. The guidewire 30 acts as a safety device during the procedure and an advantage to this approach is that the user will not lose access to the vessel access site or opening 62 in case the tensile member 250 and suture knot 264 do not seal the opening 62. If the tensile member 250 and suture knot 264 do not seal the opening 62, the introducer sheath 20 can be re-inserted over the guidewire 30 and through the opening 62. The suturing device 100 can be re-inserted into the introducer sheath 20 to deploy a second tensile member 250 and suture knot 264 to close and seal the opening 62 as described above.

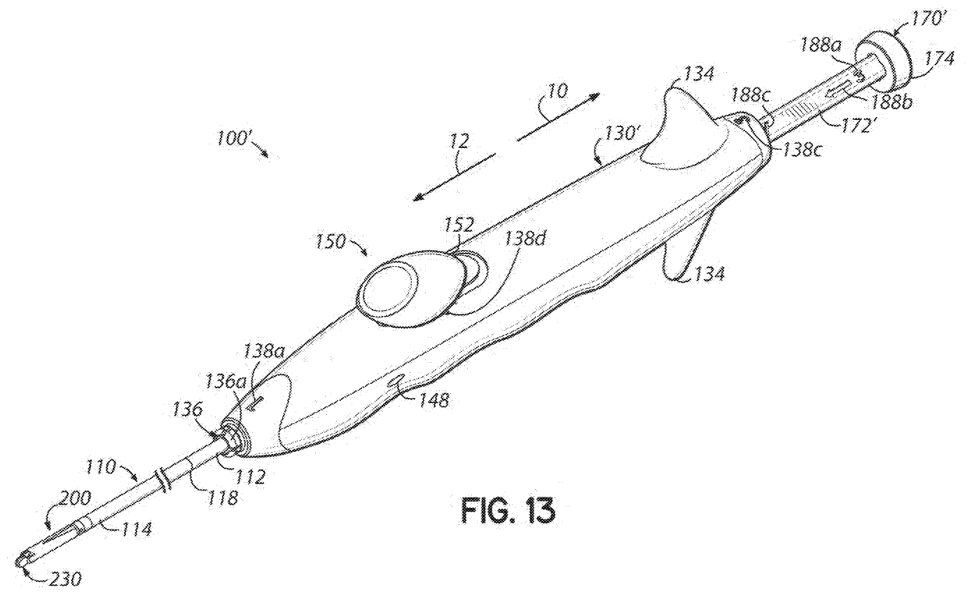

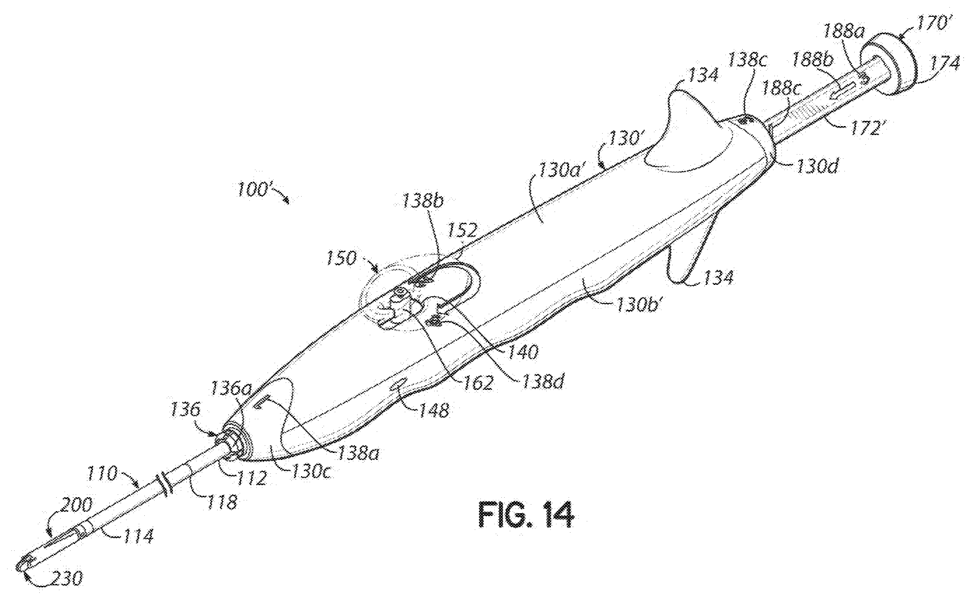

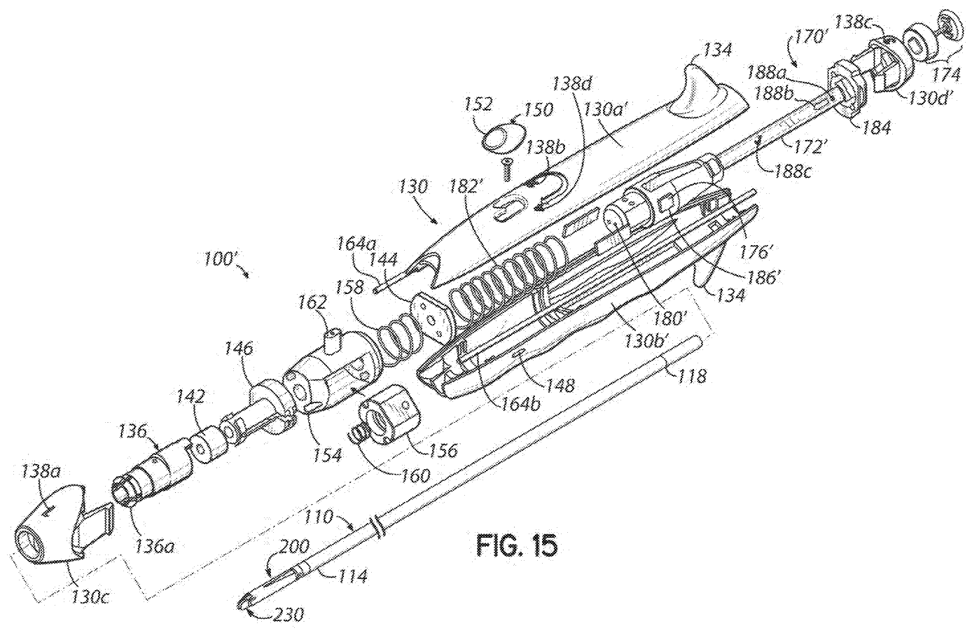

FIGS. 13 and 14 are perspective views of an alternative illustrative suturing device 100′. FIG. 15 is an exploded view of the suturing device 100′. Generally, the suturing device 100′ is similar in construction and operation to the suturing device 100 described above and the suturing device 100′ may be substituted for other suturing devices, or any feature of the suturing device 100′ may be used, in various other exemplary embodiments according to the present disclosure. Like reference numbers refer to like components. For brevity, the following description minimizes redundant description and focuses on the differences between the suturing device 100 and the suturing device 100′.

The suturing device 100′ includes an elongated shaft 110, a handle 130′ coupled to the proximal end portion 112 of the shaft 110 and a suturing mechanism 200 coupled to the distal end portion 114 of the shaft 110. The differences between the suturing device 100′ and the suturing device 100 are substantively limited to the handle 130′ and the needle actuator 170′ as described in detail below.

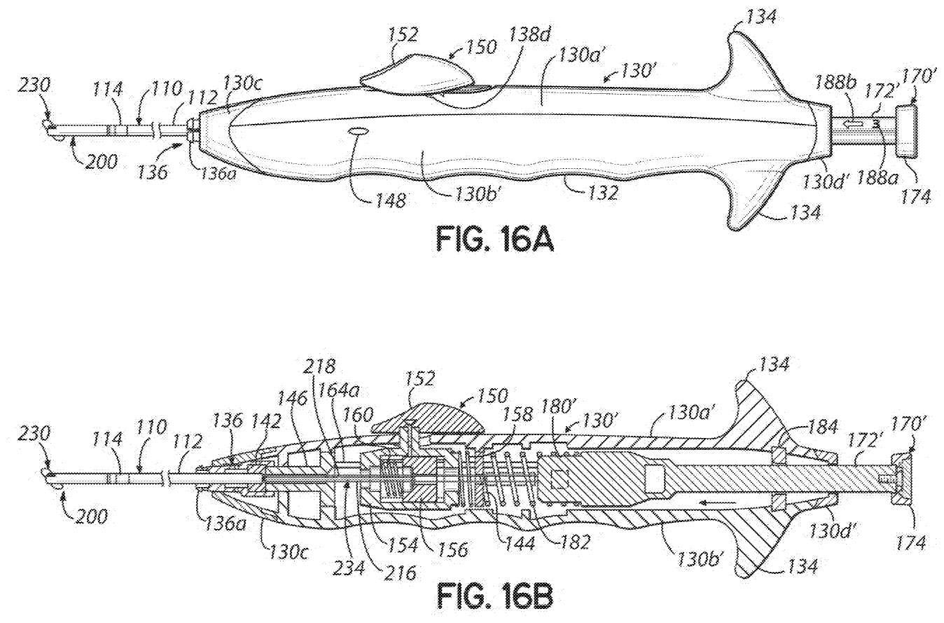

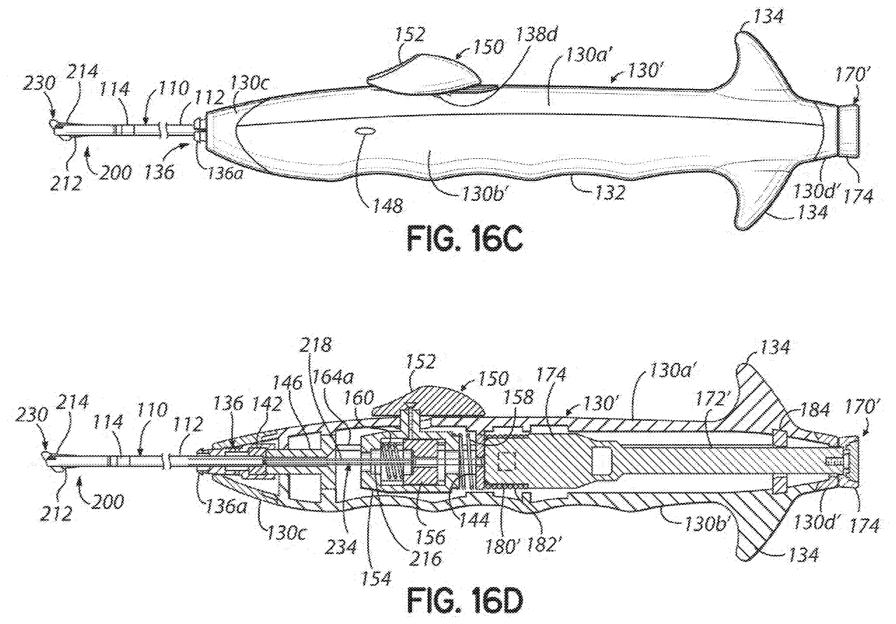

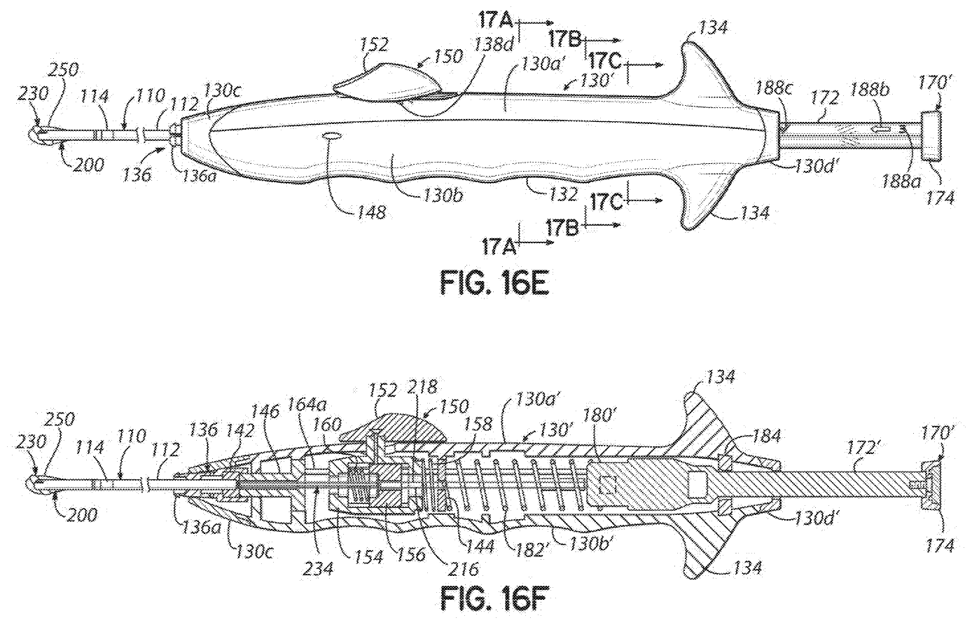

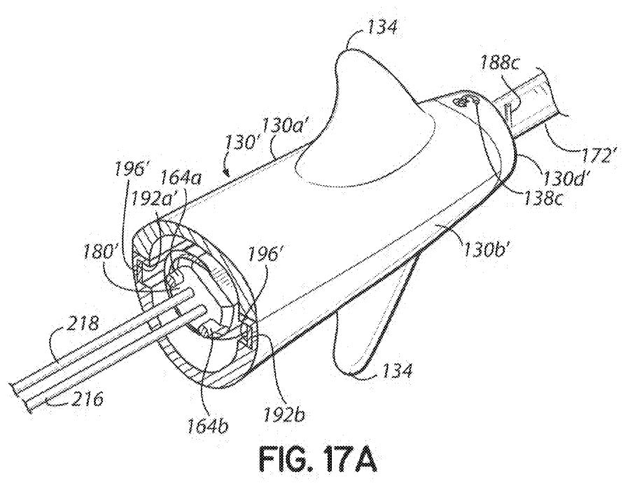

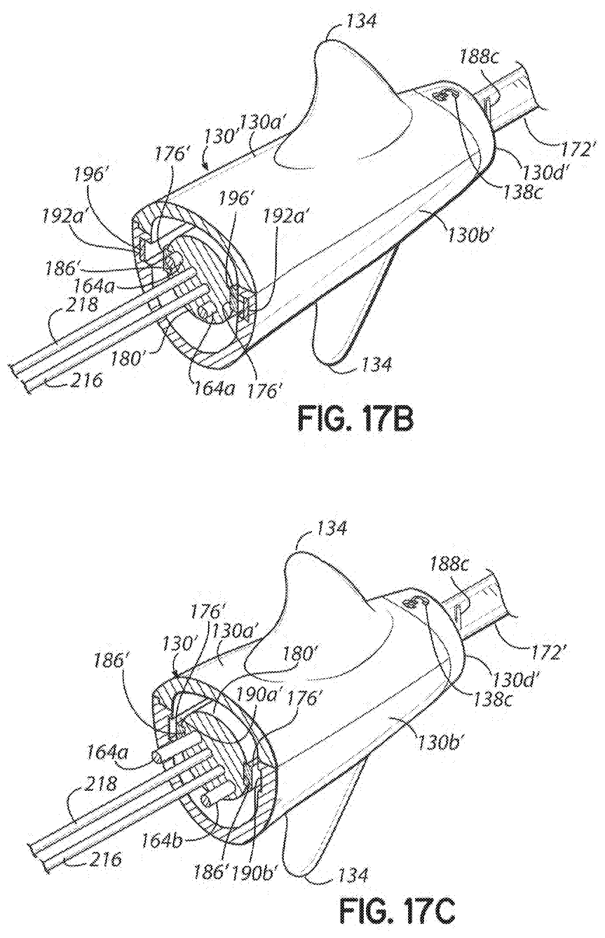

FIG. 15 shows at least some of the components of the illustrative handle 130′. FIGS. 16A, 16C, and 16E are side views of the illustrative suturing device 100′ showing at least some of the external features of the handle 130′. FIGS. 16B, 16D, and 16F are sectional side views of the suturing device 100′ showing at least some of the components of the handle 130′. FIGS. 17A, 17B, and 17C are sectional views of the handle 130′.

Referring to FIGS. 13, 15, and 16A, the handle 130′ includes an upper shell 130a′, a lower shell 130b′, a nosecone 130c, and a rear cap 130d. The suturing mechanism actuator 150 configured to operate the suturing mechanism 200 is installed in the handle 130′. A needle actuator 170′ is located at the proximal end of the handle 130′ and configured to deploy and retract the suturing needles 212, 214, see FIG. 9A.

The needle actuator 170′ includes a plunger 172′, a plunger cap 174, a pair of magnetic catches 176′, a spring 182′, and a retainer 184. Other forms of biasing members may be used in place of spring 182′. The catches 176′ are movably installed in a pair of recesses 186′ in the plunger 172′. The catches 176′ alternately engage a pair of catch grooves 190a′, 190b′ and a pair of catch recesses 192a′, 192b′ connected to catch grooves 190a′, 190b′ inside the handle 130′, see FIGS. 17A, 17B, and 17C.

The plunger 172′ includes markings 188a, 188b, 188c, to assist and instruct a user operating the various features of the suturing device 100′. While the needle actuator 170′ is shown and described as manually driven, the needle actuator 170′ may be motorized, mechanically leveraged, or assisted in other manners. Wires 216, 218 are coupled to the distal end 180′ of the plunger 172′, see FIG. 17A.

Similar to the suturing device 100 described above, FIGS. FIGS. 12A through 12M progressively illustrate the use of the suturing device 100′ and, particularly, the distal end and the suturing mechanism 200 within a blood vessel 60. Referencing FIG. 12E, with the pivotal element 230 in the deployed orientation, suture needles 212, 214 are deployed or moved in a distal direction by pushing the plunger cap 174, and thereby the plunger 172′ of the needle actuator 170′, in a distal direction as indicated by the arrow 188b, see FIGS. 16A and 16C and against the bias provided by spring 182′ see FIGS. 16B and 16D. The plunger 172′ moves suture needles 212, 214 in a distal direction toward the coupling member 270 attached to the pivotal element 230. Needle tip 212a pulls tensile member 250 distally and out of the tensile member groove 252 and then engages and connects with the first end 272 of coupling member 270, see FIG. 12F. Needle tip 214a engages and connects with the second end 274 of coupling member 270.

As the needle actuator 170′ moves in the distal direction, see FIGS. 16E and 16F the magnetic catches 176′ move in a distal direction in the catch grooves 190a′, 190b′ inside the handle 130′, see FIGS. 17A, 17B, and 17C. When the magnetic catches 176′ reach the distal end of the catch grooves 190a′, 190b′, the magnetic catches 176′ are attracted to the ferromagnetic elements 196′ which draw the magnetic catches 176′ out of the recesses 186′ in the plunger 172′ and into the catch recesses 192a', 192b′ in the handle 130′. When the magnetic catches 176′ are in the catch recesses 192a', 192b′ in the handle 130′ the plunger 172′ is free to move in the proximal direction. Some embodiments may have a single magnetic catch 176′ and ferromagnetic element 196′ while other embodiments may have a plurality of magnetic catches 176′ and ferromagnetic elements 196′.

Releasing the plunger cap 174, and thereby the plunger 172′, allows the bias of spring 182′ to move the plunger 172′ in the proximal direction, see FIGS. 16E and 16F. This action pulls the suture needles 212, 214 in a proximal direction, see FIG. 12G. As described above, suture needle 214 pulls coupling member 270 through the passage 244 of the pivotal element 230 and through the vessel wall 60a. As coupling member 270 is pulled by suture needle 214, needle tip 212a is detached from needle body 212b of suture needle 212. Needle tip 212a is pulled by coupling member 270 and pulls tensile member 250 through the vessel wall 60a.

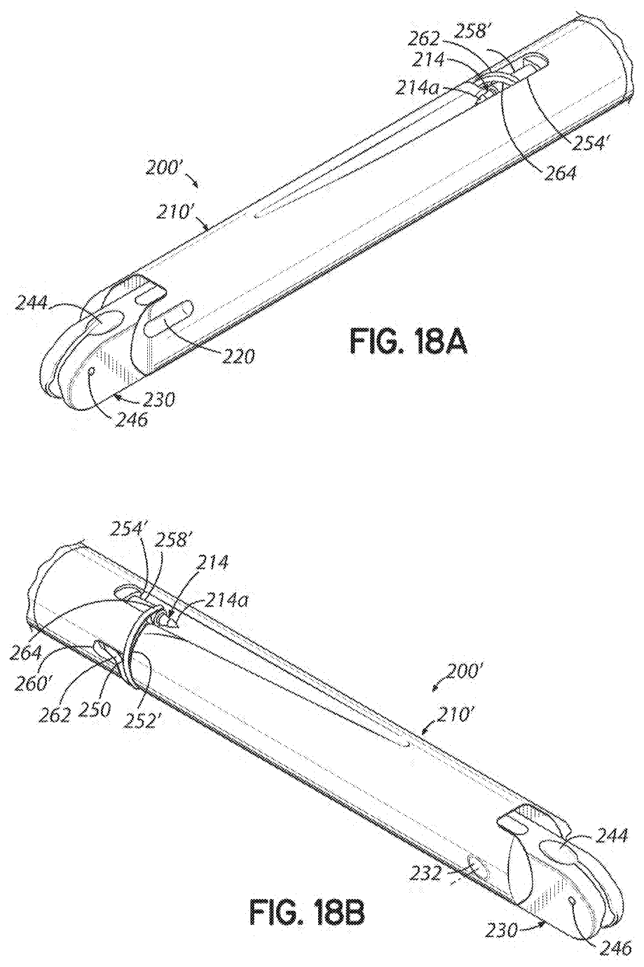

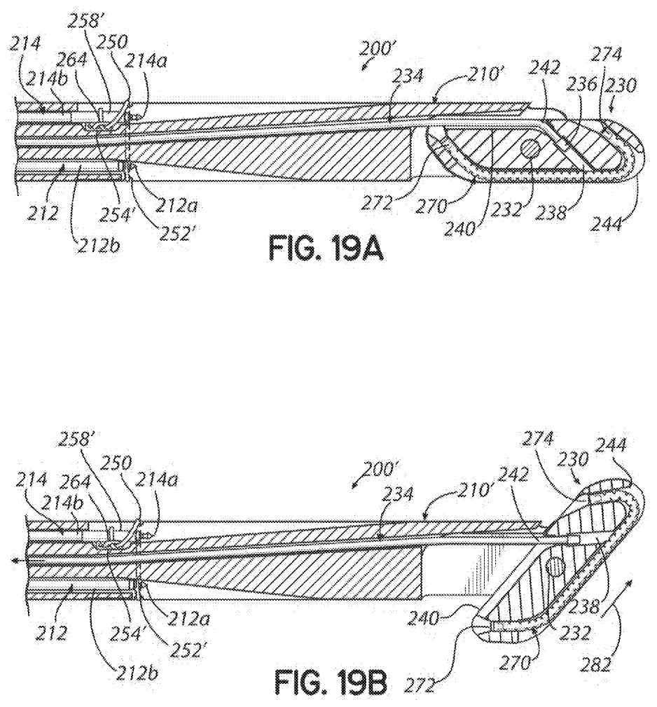

FIGS. 18A and 18B are perspective views of an alternative illustrative suturing mechanism 200′. FIGS. 19A and 19B are sectional views of the suturing mechanism 200′. Generally, the suturing mechanism 200′ is similar in construction and operation to the suturing mechanism 200 described above and the suturing mechanism 200′ may be substituted for other suturing mechanisms, or any feature of the suturing mechanism 200′ may be used, in various other exemplary embodiments according to the present disclosure. Like reference numbers refer to like components. For brevity, the following description minimizes redundant description and focuses on the differences between the suturing mechanism 200 and the suturing mechanism 200′.

In this illustrative embodiment, the suturing mechanism 200′ includes a needle guide 210′ coupled to the distal end portion 114 of the shaft 110. The needle guide 210′ includes a first needle 212, a second needle 214, and a pivotal element 230. A tensile member 250 wraps around the needle guide 210′ from the first needle 212 side to the second needle 214 side in the tensile member groove 252′. The needle guide 210′ also includes a knot recess 254′, a suture knot sleeve 258′, and slot 260′. The “coils” of the suture knot 264 are wrapped around the suture knot sleeve 258′ in the knot recess 254′. The second needle 214 passes through the suture knot sleeve 258′. The suture knot sleeve 258′ holds the suture knot 264 in the knot recess 254′ until the suture knot 264 needs to be deployed. The slot 260′ is configured to hold the free end 262 or any portion of the tensile member 250. The free end 262 of the tensile member 250 may extend from 0.13″ to 1.00″ from the suture knot 264.

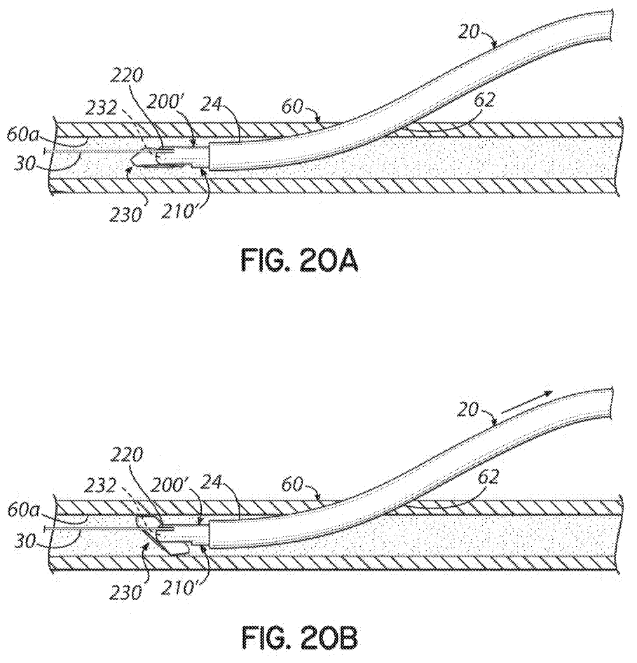

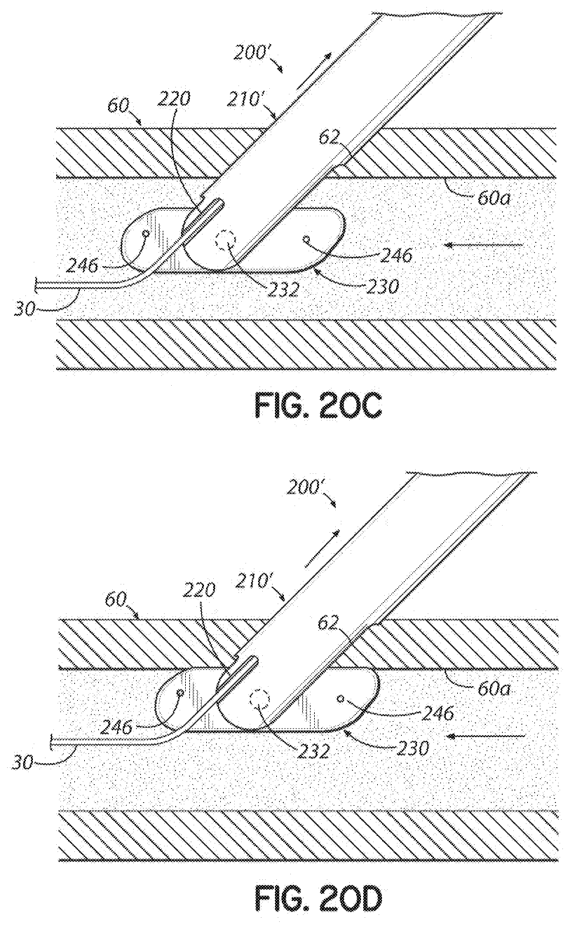

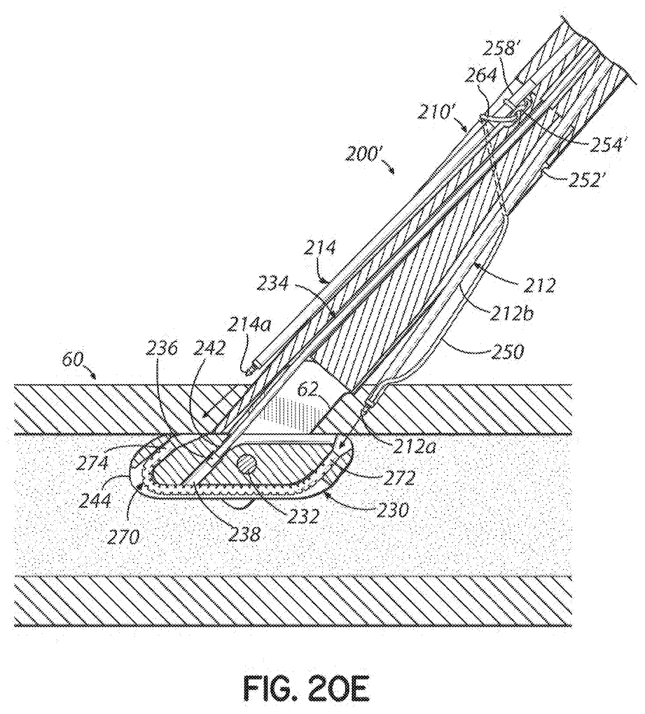

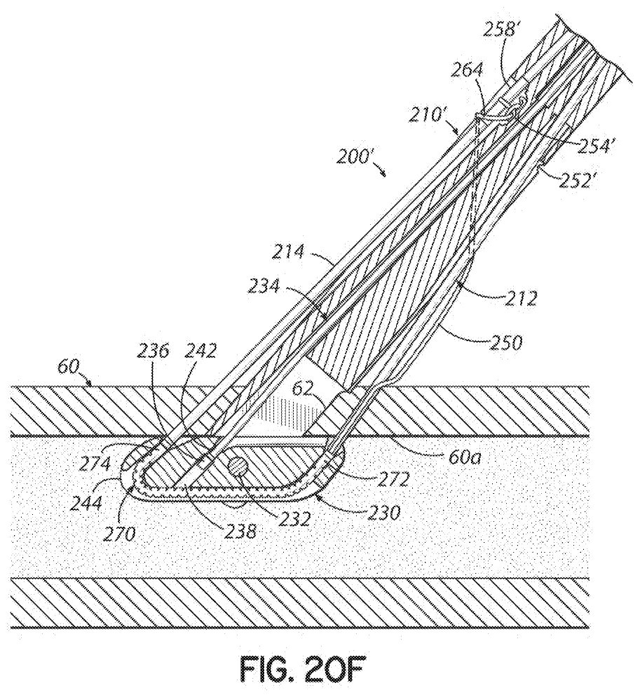

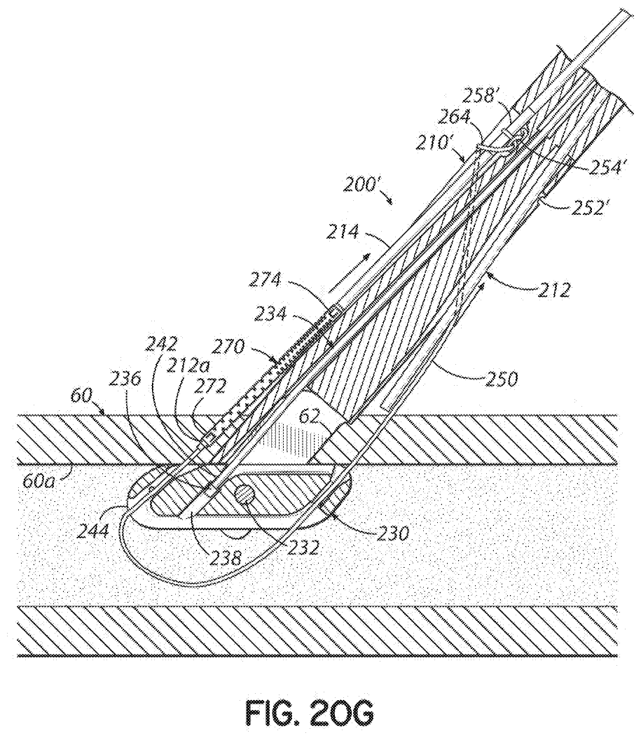

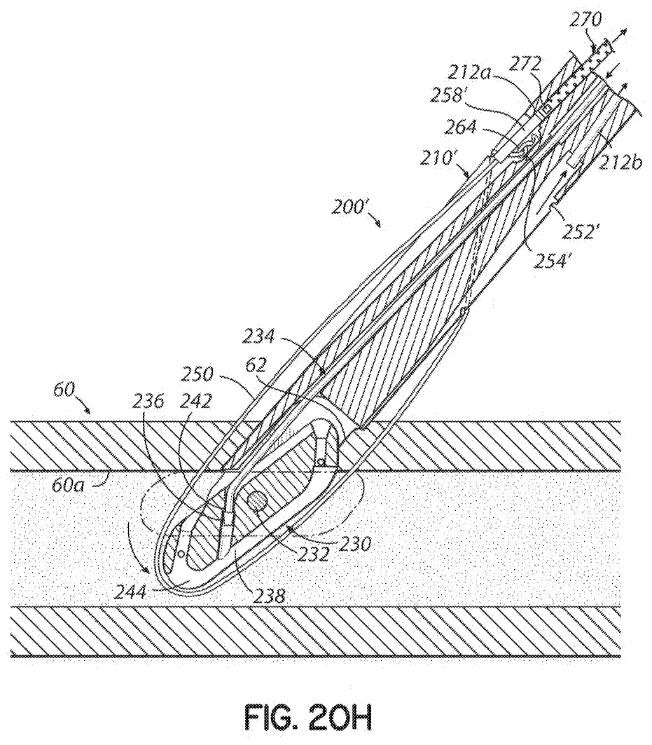

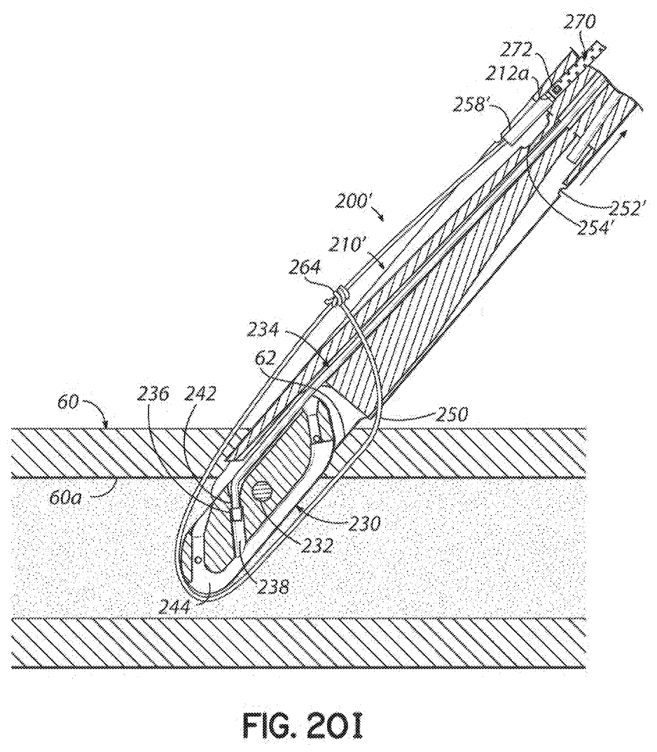

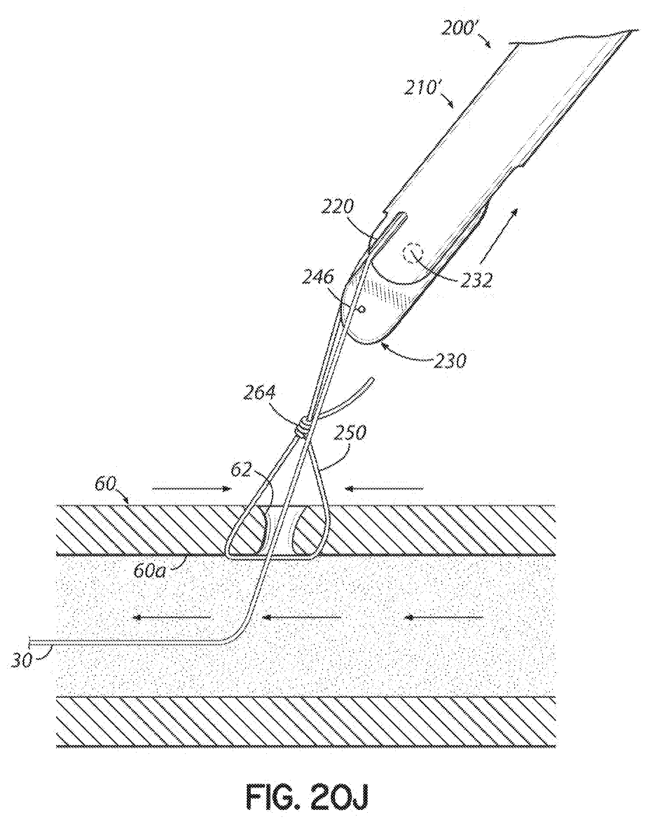

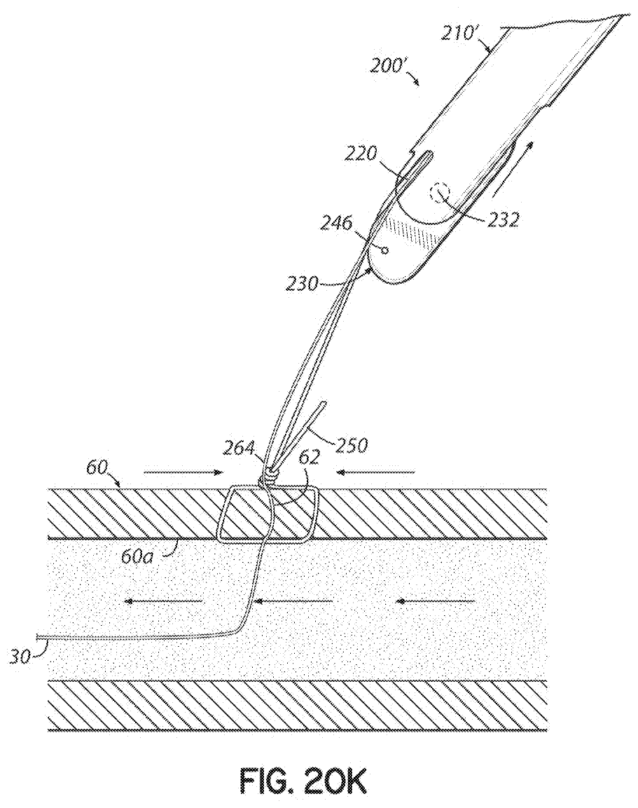

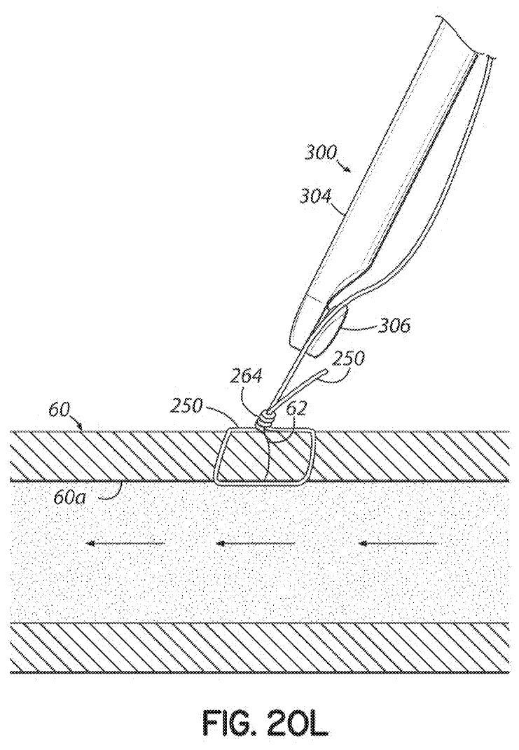

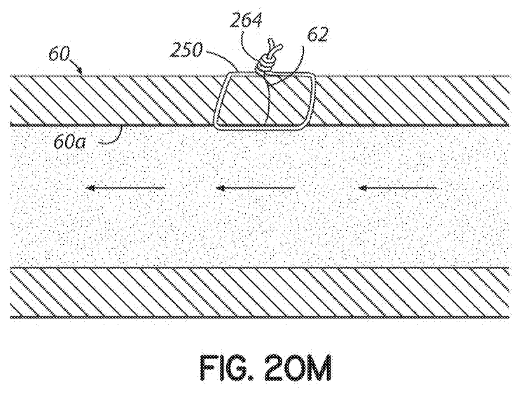

FIGS. 20A through 20M progressively illustrate the use of the suturing mechanism 200′ within a blood vessel 60, such as the common carotid artery, for closing a puncture or opening 62 in a vessel wall 60a of the blood vessel 60. The opening 62 may be a vessel access site for a medical procedure.

In preparation for a procedure, a guidewire 30, such as a 0.018″ OD guidewire, for example, is inserted in through the guidewire port 148 in the handle 130 and out through the guidewire lumen 220 in the suturing mechanism 200′, see FIGS. 2 and 8A. Alternatively, the guidewire 30 may be inserted in through the guidewire lumen 220 in the suturing mechanism 200′ and out through the guidewire port 148 in the handle 130. The guidewire 30 and the suturing mechanism 200′, with the pivotal element 230 in the insertion and removal orientation, is inserted into the proximal end 22 of the introducer sheath 20, see FIG. 1. The introducer sheath 20 is guided through the opening 62 in the blood vessel 60 using the guidewire 30.

After the distal end 24 of the introducer sheath 20 is inside the blood vessel 60, the suturing mechanism 200′ and the shaft 110 are held stationary relative to the vessel 60 and the introducer sheath 20 is pulled proximally over suturing mechanism 200′ and shaft 110 until the locking tabs 136a engage the proximal end 22 of the introducer sheath 20, see FIGS. 1 and 2 and the suturing mechanism 200′ extends past the distal end 24 of the introducer sheath 20 and into the blood vessel 60, see FIG. 20A.

With the suturing mechanism 200′ inside the blood vessel 60, the pivotal element 230 is pivoted or rotated from the insertion and removal orientation, see FIG. 19A, to the deployed orientation, see FIG. 19B. The pivotal element 230 may be rotated into the deployed orientation before the introducer sheath 20 is withdrawn from the opening 62 in the blood vessel 60, see FIG. 20B, or after the introducer sheath 20 is withdrawn from the opening 62 in the blood vessel 60, see FIG. 20C. The pivotal element 230 is then pulled proximally against the interior of the vessel wall 60a as shown in FIG. 20D.

While the pivotal element 230 is in the deployed orientation, suture needles 212, 214 are deployed or moved in a distal direction, see FIG. 20E. Needle tip 212a pulls tensile member 250 distally and out of the tensile member groove 252′ and then engages and connects with the first end 272 of coupling member 270, see FIG. 20F. Needle tip 214a engages and connects with the second end 274 of coupling member 270. The suture needles 212, 214 are pulled in a proximal direction, see FIG. 20G. Suture needle 214 pulls coupling member 270 through the passage 244 of the pivotal element 230 and through the vessel wall 60a. As coupling member 270 is pulled by suture needle 214, needle tip 212a is detached from needle body 212b of suture needle 212. Needle tip 212a is pulled by coupling member 270 and pulls tensile member 250 through the vessel wall 60a. Suture needle 214 pulls coupling member 270, needle tip 212a, and tensile member 250 through the suture knot sleeve 258′, see FIG. 20I.

The pivotal element 230 is moved distally away from the interior of the vessel wall 60a and then rotated from the deployed orientation to the insertion and removal orientation, see FIG. 20H. With the pivotal element 230 in the insertion and removal orientation the suturing mechanism 200′ is pulled in the proximal direction thereby removing the suturing mechanism 200′ from the blood vessel 60 and releasing the suture knot 264 from the suture knot sleeve 258′ and the knot recess 254′, see FIG. 20I. As the suturing mechanism 200′ is pulled in the proximal direction the suture knot 264 slides along the tensile member 250 towards the opening 62 in the blood vessel 60. As the suturing mechanism 200′ is pulled proximally, the suture knot 264 is tightened against the outside of the vessel 60, see FIGS. 20J and 20K, to approximate or close the opening 62. The guidewire 30 remains in the opening 62 while the suture knot 264 is tightened against the outside of the vessel 60 and the guidewire 30 is not removed until the opening 62 is closed. During some procedures it may be necessary to push the suture knot 264 toward the opening 62. Referring to FIGS. 20L and 12N, a knot pusher 300, including a handle 302, an elongated shaft 304, and loop or ring 306, may be used to push the suture knot 264 toward the opening 62. Finally, the ends of tensile member 250 outside of the suture knot 264 are cut off, leaving the opening 62 securely closed and sealed as shown in FIG. 20M. The guidewire 30 acts as a safety device during the procedure and an advantage to this approach is that the user will not lose access to the vessel access site or opening 62 in case the tensile member 250 and suture knot 264 do not seal the opening 62. If the tensile member 250 and suture knot 264 do not seal the opening 62, the introducer sheath 20 can be re-inserted over the guidewire 30 and through the opening 62. The suturing device 100 can be re-inserted into the introducer sheath 20 to deploy a second tensile member 250 and suture knot 264 to close and seal the opening 62 as described above.

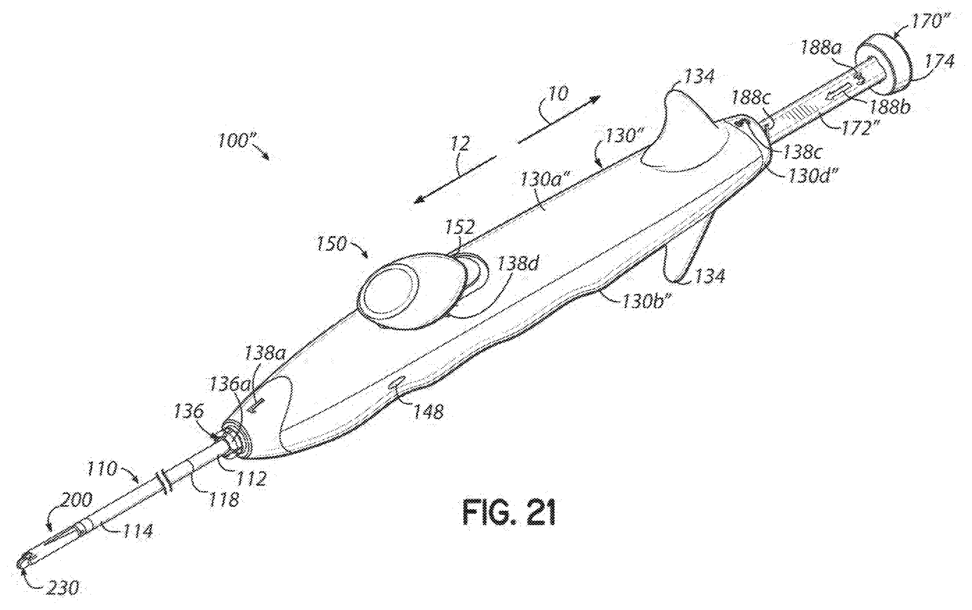

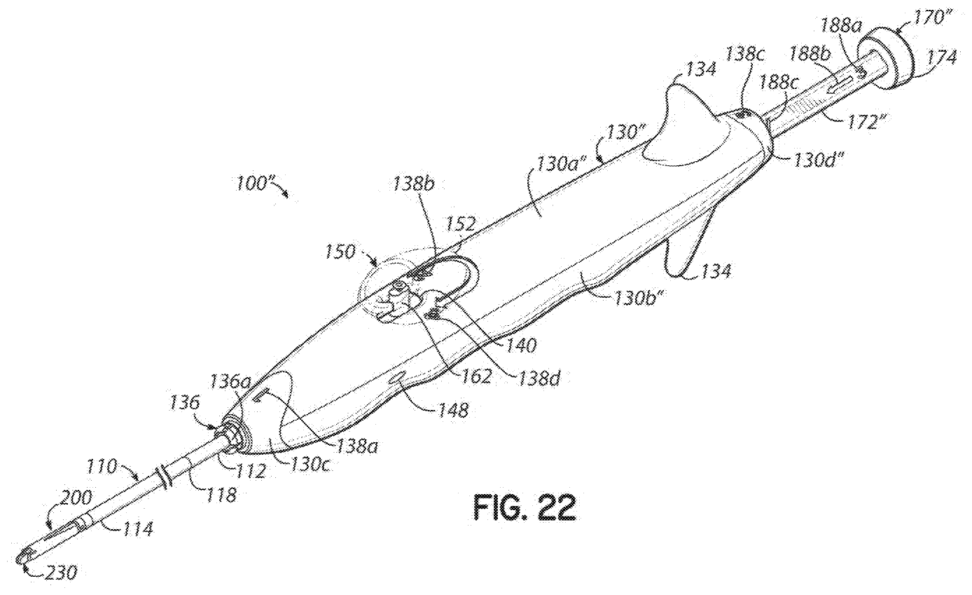

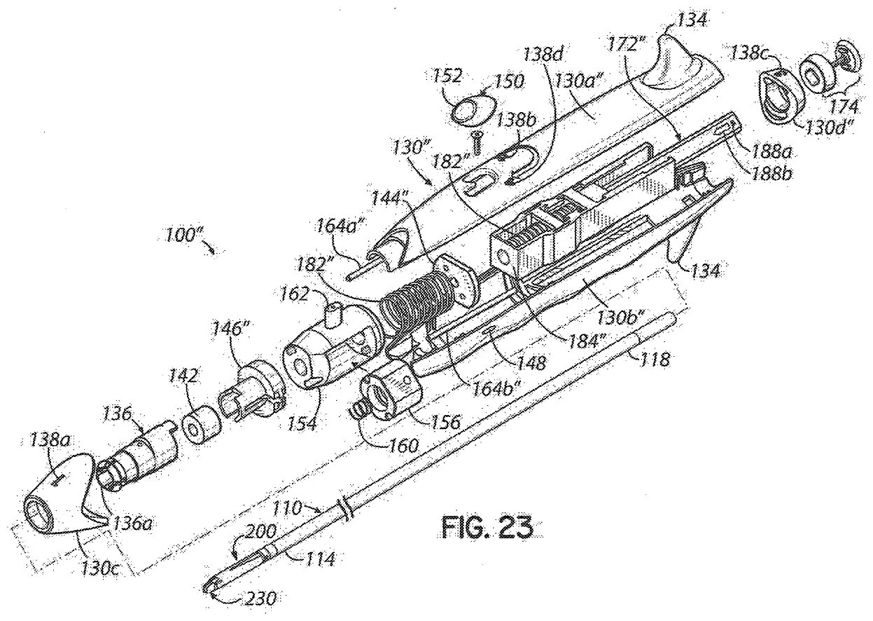

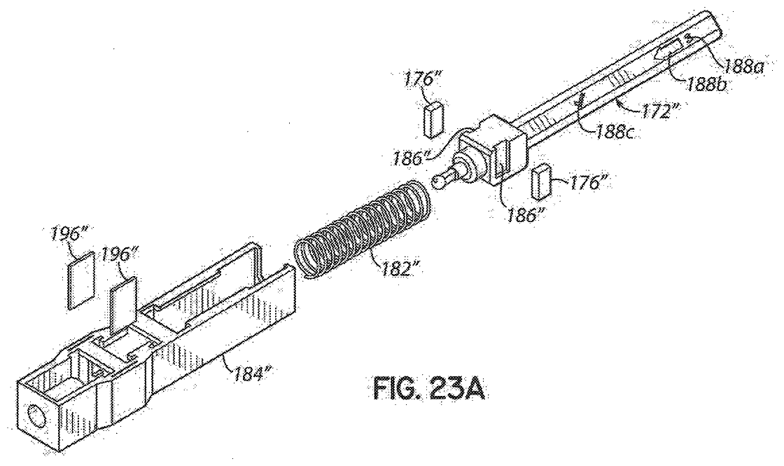

FIGS. 21 and 22 are perspective views of an alternative illustrative suturing device 100″. FIG. 23 is an exploded view of the suturing device 100″. Generally, the suturing device 100″ is similar in construction and operation to the suturing devices 100 and 100′ described above and the suturing device 100′ may be substituted for other suturing devices, or any feature of the suturing device 100′ may be used, in various other exemplary embodiments according to the present disclosure. Like reference numbers refer to like components. For brevity, the following description minimizes redundant description and focuses on the differences between the suturing devices 100 and 100″ and the suturing device 100′.

The suturing device 100″ includes an elongated shaft 110, a handle 130″ coupled to the proximal end portion 112 of the shaft 110 and a suturing mechanism 200 coupled to the distal end portion 114 of the shaft 110. The differences between the suturing device 100″ and the suturing devices 100 and 100′ are substantively limited to the handle 130″ and the needle actuator 170″ as described in detail below.

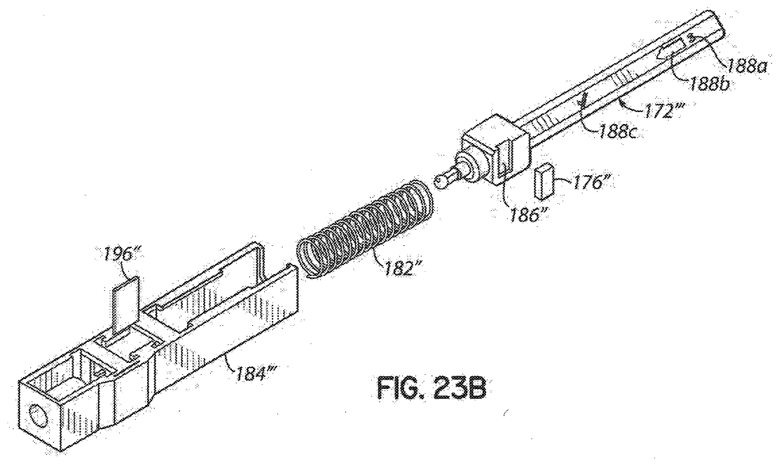

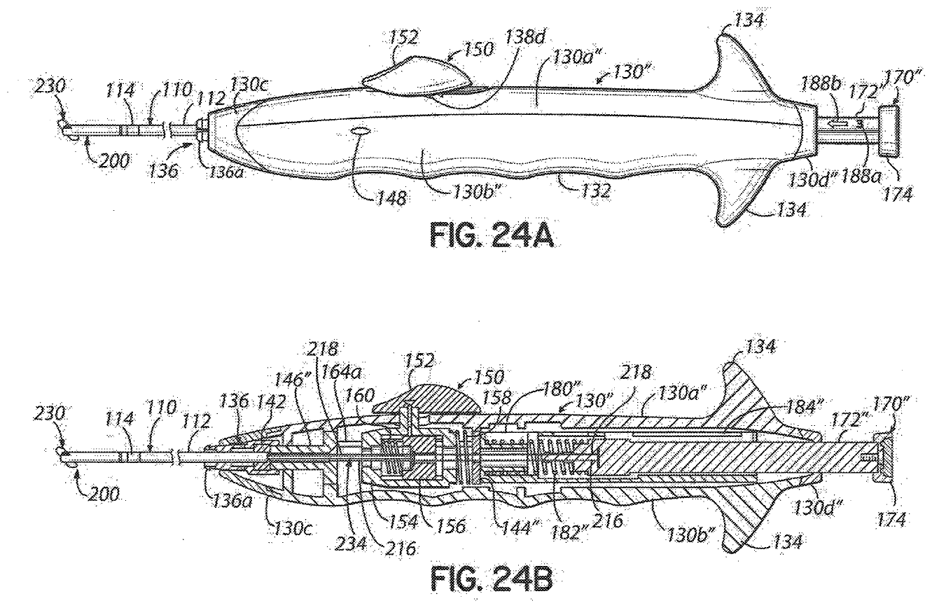

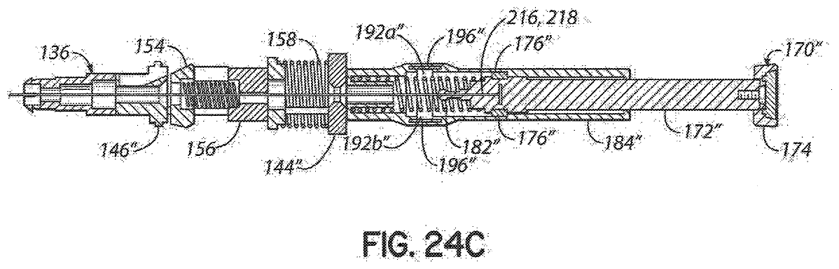

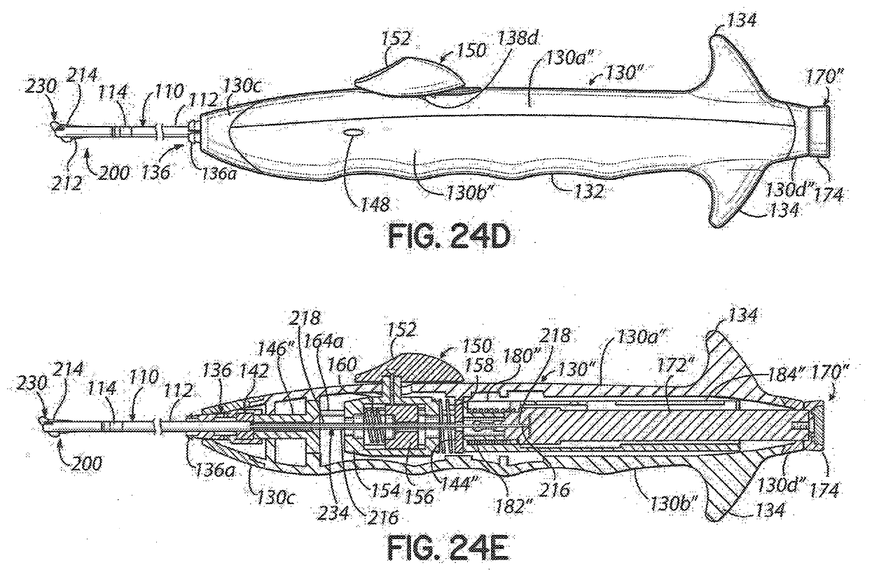

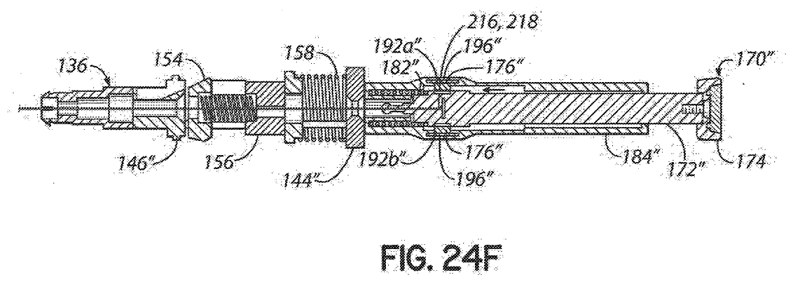

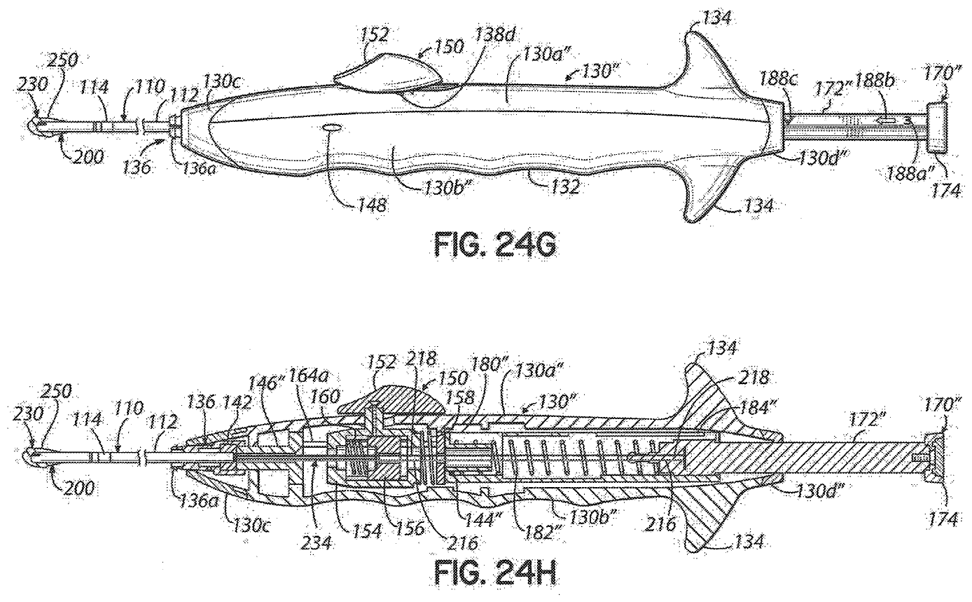

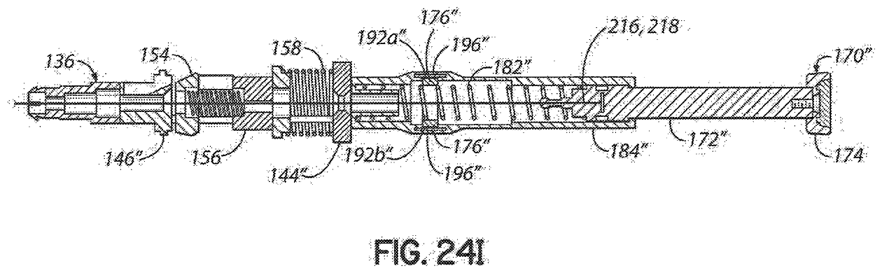

FIG. 23 is an exploded view of the suturing device 100″ and shows at least some of the components of the illustrative handle 130″. FIG. 23A is an exploded view of a portion of the handle 130″ of the suturing device 100″ FIGS. 24A, 24D, and 24G are side views of the illustrative suturing device 100″ showing at least some of the external features of the handle 130″. FIGS. 24B, 24E, and 24H are sectional side views of the suturing device 100″ showing at least some of the components of the handle 130″. FIGS. 24C, 24F, and 24I are sectional top views a portion of the handle 130″ of the suturing device 100″ showing at least some of the components of the handle 130″.

Referring to FIGS. 21, 23, and 23A, the handle 130″ includes an upper shell 130a″, a lower shell 130b″, a nosecone 130c, and a rear cap 130d″. The suturing mechanism actuator 150 configured to operate the suturing mechanism 200 is installed in the handle 130″. A needle actuator 170″ is located at the proximal end of the handle 130″ and configured to deploy and retract the suturing needles 212, 214, see FIG. 9A.

The needle actuator 170″ includes a plunger 172″, a plunger cap 174, one or more magnetic catches 176″, a spring 182″, and a linear retainer 184″. Other forms of biasing members may be used in place of spring 182″. The catches 176″ are movably installed in a pair of recesses 186″ in the plunger 172″. The catches 176″ alternately engage a pair of catch grooves 190a“, 190b” and one or more catch recesses 192a', 192b′ connected to one or more catch grooves 190a′, 190b′ inside the linear retainer 184″, see FIGS. 24C, 24F, and 241. The plunger 172″ includes markings 188a, 188b, 188c, to assist and instruct a user operating the various features of the suturing device 100″. While the needle actuator 170″ is shown and described as manually driven, the needle actuator 170″ may be motorized, mechanically leveraged, or assisted in other manners. Wires 216, 218 are coupled to the distal end 180″ of the plunger 172″.

Similar to the suturing device 100 described above, FIGS. 12A through 12M progressively illustrate the use of the suturing device 100″ and, particularly, the distal end and the suturing mechanism 200 within a blood vessel 60. Referencing FIG. 12E, with the pivotal element 230 in the deployed orientation, suture needles 212, 214 are deployed or moved in a distal direction by pushing the plunger cap 174, and thereby the plunger 172″ of the needle actuator 170″, in a distal direction as indicated by the arrow 188b, see FIGS. 24A and 24D and against the bias provided by spring 182″, see FIGS. 24B and 24C. The plunger 172″ moves suture needles 212, 214 in a distal direction toward the coupling member 270 attached to the pivotal element 230. Needle tip 212a pulls tensile member 250 distally and out of the tensile member groove 252 and then engages and connects with the first end 272 of coupling member 270, see FIG. 12F. Needle tip 214a engages and connects with the second end 274 of coupling member 270.

As the needle actuator 170″ moves in the distal direction, the magnetic catches 176″ move in a distal direction in the catch grooves 190a″, 190b″ inside the linear retainer 184″, see FIGS. 24B and 24C. When the magnetic catches 176′ reach the distal end of the catch grooves 190a′, 190b′, the magnetic catches 176′ are attracted to the ferromagnetic elements 196″ which draw the magnetic catches 176″ out of the recesses 186″ in the plunger 172″ and into the catch recesses 192a″, 192b″ in linear retainer 184″, see FIGS. 24D, 24E, and 24F. When the magnetic catches 176″ are in the catch recesses 192a″, 192b″ in the linear retainer 184″ the plunger 172″ is free to move in the proximal direction. Some embodiments may have a single magnetic catch 176″ and ferromagnetic element 196″ while other embodiments may have a plurality of magnetic catches 176″ and ferromagnetic elements 196″, see FIG. 23B.

Releasing the plunger cap 174, and thereby the plunger 172″, allows the bias of spring 182″ to move the plunger 172″ in the proximal direction, see FIGS. 24G, 24H, and 24I . This action pulls the suture needles 212, 214 in a proximal direction, see FIG. 12G. As described above, suture needle 214 pulls coupling member 270 through the passage 244 of the pivotal element 230 and through the vessel wall 60a. As coupling member 270 is pulled by suture needle 214, needle tip 212a is detached from needle body 212b of suture needle 212. Needle tip 212a is pulled by coupling member 270 and pulls tensile member 250 through the vessel wall 60a.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination within and between the various embodiments. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept.

What is claimed is:

1. A suture device comprising:
a tensile member;
a suture knot associated with the tensile member; and
a needle guide including:
a first needle coupled to the tensile member,
a second needle, and
a suture knot recess configured to accept the suture knot;
wherein at least a portion of the suture device is configured to be directed into an opening in a tissue,
wherein the first and second needles are configured to be directed through the tissue adjacent to the opening to direct the tensile member through the tissue,
wherein the tensile member is configured to be pulled through the tissue when at least one of the first or second needles is retracted, and
wherein the suture knot is configured to be deployed from the suture knot recess and along the tensile member and tightened to close and seal the opening.

2. The suture device of claim 1, wherein the suture knot is deployed when at least one of the first or second needles is retracted.

3. The suture device of claim 1, wherein the tensile member is drawn through the suture knot when at least one of the first or second needles is retracted.

4. The suture device of claim 1, further comprising a suture knot sleeve;
wherein the suture knot is wrapped around the suture knot sleeve and the suture knot sleeve retains the suture knot until the suture knot is deployed.

5. The suture device of claim 4, wherein the suture knot sleeve is at least partially inside the suture knot recess.

6. The suture device of claim 1, wherein the needle guide further comprises a tensile member groove configured to accommodate the tensile member;
wherein the tensile member is configured to be deployed from the tensile member groove when at least one of the first or second needles is directed through the tissue.

7. The suture device of claim 6, wherein the tensile member groove extends from a first needle side to a second needle side of the needle guide.

8. The suture device of claim 1, wherein the suture knot is a sliding knot.

9. The suture device of claim 1, wherein the suture knot is a “Tennessee Slider” knot.

10. The suture device of claim 1, further comprising:
a handle located at a proximal end portion of the suture device;
an elongated shaft coupled between the handle and the needle guide; and
a needle actuator located on the handle, coupled to at least one of the first or second needles, and configured to direct at least one of the first or second needles through the tissue when the needle actuator is activated.

11. The suture device of claim 10, wherein the needle actuator includes a biasing member configured to retract at least one of the first or second needles.

12. The suture device of claim 11, wherein the needle actuator biasing member is a spring.

13. The suture device of claim 10, wherein the needle actuator is coupled to the first and second needles,
the needle actuator is in a first needle actuator position prior to directing the first and second needles through the tissue,
the needle actuator is configured to be moved to a second needle actuator position to direct the first and second needles through the tissue, and
the needle actuator is configured to be moved to a third needle actuator position to retract the first and second needles.

14. The suture device of claim 13, wherein the needle actuator comprises:
a plunger; and
a biasing member;
wherein when the plunger is depressed the needle actuator moves from the first needle actuator position to the second needle actuator position, and
wherein when the plunger is released the biasing member moves the needle actuator from the second needle actuator position to the third needle actuator position.

15. The suture device of claim 14, wherein the needle actuator biasing member is a spring.

16. The suture device of claim 14, wherein the needle actuator further comprises a sprag;
wherein when the plunger is depressed the sprag moves from a first sprag orientation to a second sprag orientation, and
wherein when in the second sprag orientation, the sprag allows the needle actuator to move from the second needle actuator position to the third needle actuator position when the plunger is released.

17. The suture device of claim 16, wherein the sprag is configured to rotate from the first sprag orientation to the second sprag orientation.

18. The suture device of claim 16, wherein the sprag engages a first sprag groove when the needle actuator is in the first needle actuator position, and
wherein the sprag engages a second sprag groove when the plunger is depressed thereby allowing the needle actuator to move from the second needle actuator position to the third needle actuator position when the plunger is released.

19. The suture device of claim 14, wherein the needle actuator further comprises a catch;
wherein when depressed, the plunger moves the catch, and
wherein when moved, the catch allows the needle actuator to move from the second needle actuator position to the third needle actuator position when the plunger is released.

20. The suture device of claim 19, wherein at least a portion of the catch is magnetic.
